(19) United States
(12) Patent Application Publication
(10) Pub. No.: US 2013/0105786 A1
(43) Pub. Date: May 2, 2013

(54) NOVEL SPIRO(ANTHRACENE-9,9'-FLUOREN)-10-ONE COMPOUND AND ORGANIC LIGHT-EMITTING DEVICE INCLUDING THE SAME

(71) Applicants: Taiki Watanabe, Akishima-shi (JP); Masashi Hashimoto, Tokyo (JP); Jun Kamatani, Tokyo (JP); Akihito Saitoh, Gotemba-shi (JP)

(72) Inventors: Taiki Watanabe, Akishima-shi (JP); Masashi Hashimoto, Tokyo (JP); Jun Kamatani, Tokyo (JP); Akihito Saitoh, Gotemba-shi (JP)

(73) Assignee: CANON KABUSHIKI KAISHA, Tokyo (JP)

(21) Appl. No.: 13/809,402

(22) PCT Filed: Jul. 8, 2011
(86) PCT No.: PCT/JP2011/066170

§ 371 Date: Jan. 9, 2013

(30) Foreign Application Priority Data
Jul. 13, 2010 (JP) 2010-158569

(51) Int. Cl. H01L 51/00

(52) U.S. Cl. CPC H01L 51/0052; H01L 51/0073; H01L 51/0074
USPC 257/40; 568/326; 549/330; 549/43

(57) ABSTRACT

A novel and stable spiro(anthracene-9,9-fluoren)-10-one compound represented by general formula [1] is provided.

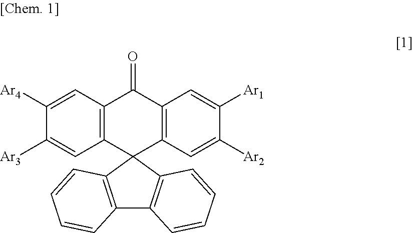

### BACKGROUND OF THE INVENTION

1. Technical Field

The present invention relates to a novel spiro(anthracene-9,9′-fluoren)-10-one compound and an organic light-emitting device including the compound.

2. Background Art

A light-emitting device is a device that includes an anode, a cathode, and an organic compound layer interposed between the anode and cathode. Holes and electrons injected from the respective electrodes of the organic light-emitting device are recombined in the organic compound layer serving as an emission layer to generate excitons and light is emitted as the excitons return to their ground state. Recent years have seen remarkable advances in the field of organic light-emitting devices. Organic light-emitting devices offer low driving voltage, various emission wavelengths, rapid response, and small thickness and are light-weight.

Organic light-emitting devices that emit phosphorescent are a type of organic light-emitting device that includes an emission layer containing a phosphorescent material, with triplet excitons contributing to emission. There is still room for improving the emission efficiency of organic light-emitting devices that emit phosphorescence.

PTL 1 discloses an invention related to an organic light-emitting device. PTL 1 discloses an anthrone (compound a) that is represented by a formula below and serves as an intermediate for synthesizing anthracene.

PTL 2 discloses a 10,10-diphenylanthrone derivative (compound b) represented by a formula below and used in a hole transport layer of a fluorescent organic light-emitting device.

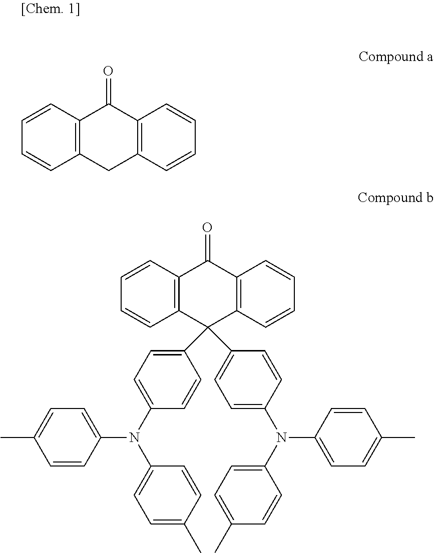

The compounds disclosed in PTL 1 and 2 have an anthrone skeleton with the 10-position substituted with hydrogen or two aryl groups. When the 10-position is substituted with hydrogen, the compound is instable because elimination of reactive hydrogen occurs and anthracene is formed. When the 10-position is substituted with two aryl groups, the stability of the basic skeleton is deteriorated because the two aryl groups not bonded with each other can rotate separately. Moreover, both PTL 1 and 2 fail to focus on and utilize the electron transport property of the anthrone skeleton.

As for organic light-emitting devices having emission layers, development of organic compounds for use in electron transport layers is sought after. In particular, a chemically stable organic compound that has a lowest unoccupied molecular orbital (LUMO) level as deep as 2.7 eV or more is desired.

As for organic light-emitting devices that contains a phosphorescent material in emission layers, an organic compound that also has a high T₁ energy that can be used in such devices is desired.

### CITATION LIST

### Patent Literature

PTL 1 Japanese Patent Laid-Open No. 2002-338957

PTL 2 Japanese Patent Laid-Open No. 08-259937

### SUMMARY OF INVENTION

### Technical Problem

The present invention provides a spiro(anthracene-9,9-fluoren)-10-one compound represented by general formula [1] below.

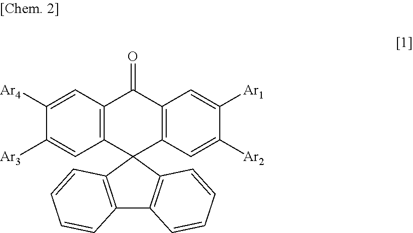

In formula [1], Ar₁ and Ar₂ each independently denote a hydrogen atom, a phenyl group, a biphenyl group, a terphenyl group, a dimethylfluorenyl group, a triphenylene group, a dibenzofuran group, or a dibenzothiophene group.

One of Ar₁ and Ar₂ denotes a hydrogen atom.

Ar₃ and Ar₄ each independently denote a hydrogen atom, a phenyl group, a biphenyl group, a terphenyl group, a dimethylfluorenyl group, a triphenylene group, a dibenzofuran group, or a dibenzothiophene group.

One of Ar₃ and Ar₄ denotes a hydrogen atom.

### Advantageous Effects of Invention

The present invention provides a novel spiro(anthracene-9,9-fluoren)-10-one compound having T₁ energy of 2.3 eV or more and a LUMO level of 2.7 eV or more. An organic light-emitting device that uses this compound achieves high emission efficiency and low driving voltage.

### BRIEF DESCRIPTION OF DRAWINGS

FIG. 1 is a cross-sectional view of an organic light-emitting device and a switching device connected to the organic light-emitting device.

### DESCRIPTION OF EMBODIMENTS

A spiro(anthracene-9,9′-fluoren)-10-one compound according to an embodiment of the invention is represented by general formula [1] below.

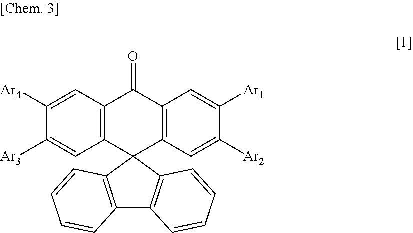

In Formula [1], Ar₁ and Ar₂ each independently denote a hydrogen atom, a phenyl group, a biphenyl group, a terphenyl group, a dimethylfluorenyl group, a triphenylene group, a dibenzofuran group, or a dibenzothiophene group.

One of Ar₁ and Ar₂ denotes a hydrogen atom.

Ar₃ and Ar₄ each independently denote a hydrogen atom, a phenyl group, a biphenyl group, a terphenyl group, a dimethylfluorenyl group, a triphenylene group, a dibenzofuran group, or a dibenzothiophene group.

One of Ar₃ and Ar₄ denotes a hydrogen atom.

In particular, the spiro(anthracene-9,9′-fluoren)-10-one compound has a structure in which an anthrone ring having substituents is joined with a fluorene ring through a spiro carbon. Possible combinations of the substitution positions on the anthrone ring are as follows:
(1) Combination of Ar₁ and Ar₃ (equivalent to the combination of Ar₂ and Ar₄)
(2) Combination of Ar₁ and Ar₄
(3) Combination of Ar₂ and Ar₃
All combinations give a compound having T₁ energy of 2.3 eV or more and a LUMO level 2.7 eV or deeper.

In the spiro(anthracene-9,9′-fluoren)-10-one compound, sites other than those substituted with Ar₁ to Ar₄, i.e., R₁ to R₁₂ in general formula [2] below, may each be substituted with a hydrogen atom or an alkyl group having 1 to 4 carbon atoms. Examples of the alkyl group having 1 to 4 carbon atoms include a methyl group, an ethyl group, an n-propyl group, an iso-propyl group, an n-butyl group, an iso-butyl group, a sec-butyl group, and a tert-butyl group. R₁ to R₁₂ are preferably each substituted with a hydrogen since the synthetic process is easy.

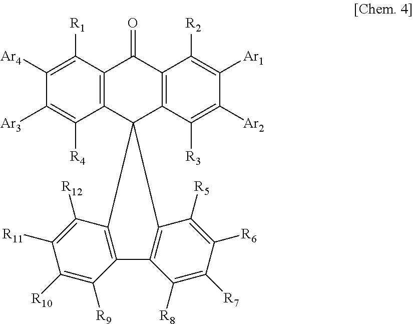

A novel stable spiro(anthracene-9,9′-fluoren)-10-one compound described herein reflects the high T₁ energy (i.e., 2.86 eV (433 nm)) and the deep LUMO level (2.7 eV or more) inherent to spiro(anthracene-9,9′-fluoren)-10-one represented by the following formula:

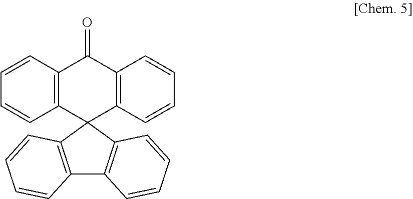 [SMILES: O=C1c2ccccc2C2(c3ccccc31)c1ccccc1-c1ccccc12]

An organic light-emitting device that uses this compound achieves high emission efficiency, low driving voltage, and stability.

The spiro(anthracene-9,9′-fluoren)-10-one compound and the light-emitting device according to embodiments of the invention will now be described in detail.

Properties of piro(anthracene-9,9′-fluoren)-10-one Compound

Properties of the spiro(anthracene-9,9′-fluoren)-10-one compound according the present invention are described in sections (1) and (2) below.

(1) The anthrone skeleton represented by the following formula has a 10-position that has high reactivity:

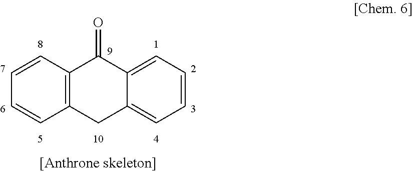 [SMILES: O=C1c2ccccc2Cc2ccccc21]

The anthrone skeleton is widely used as an intermediate for synthesizing anthracene. The reaction path for obtaining anthracene from the anthrone skeleton is as follows:

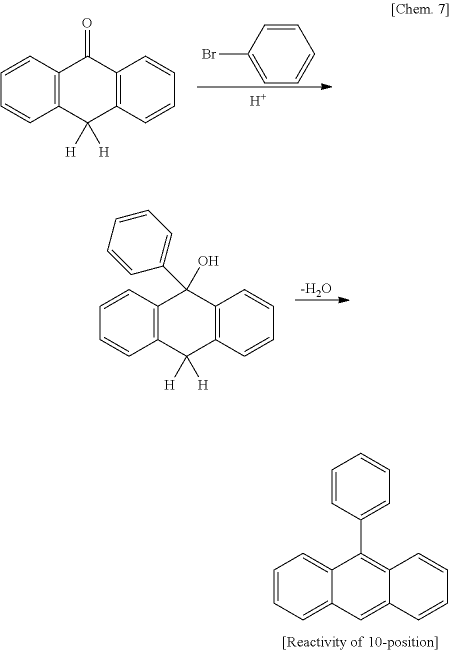

This reaction occurs because the 10-position of the anthrone skeleton are substituted with hydrogen atoms. In contrast, the spiro(anthracene-9,9′-fluoren)-10-one compound of the embodiment does not undergo the reaction represented by the scheme above and is thus stable.

(2) As for a compound having an anthrone skeleton with two aryl groups substituting the 10-position, the two aryl groups can rotate separately since they are not bonded to each other, and thus the stability of the basic skeleton is low. In contrast, according to the spiro(anthracene-9,9′-fluoren)-10-one compound of the invention, the anthrone skeleton is spiro-bonded with the fluorene skeleton at the 10-position of the anthrone skeleton. Thus, the compound has no rotatable portions and exhibits high stability. An organic light-emitting device that uses a compound having a rotatable portion is not desirable since deterioration (decrease in luminance and efficiency) with time is accelerated.

As set forth in (1) and (2) above, when the anthrone skeleton is used in an organic light-emitting device, a spiro(anthracene-9,9′-fluoren)-10-one compound may be used so that the organic light-emitting device has high stability.

Functions of the spiro(anthracene-9,9′-fluoren)-10-one Compound

The anthrone ring in the spiro(anthracene-9,9′-fluoren)-10-one compound has a carbonyl group. The inventors of the present invention have found that this compound is suitable for use in layers that confine electrons or allow electrons to flow, i.e., electron transport layers, hole blocking layers, and emission layers of organic light-emitting devices. A hole blocking layer is a layer that is adjacent to a cathode-side of an emission layer or an electron transport layer. An electron transport layer is a layer in contact with a cathode and is also called an “electron injection layer”. A hole blocking layer may also be called a “layer adjacent to a cathode-side of an emission layer or an electron transport layer”. The inventors have found that this compound is particularly suitable for use in an emission layer or a hole blocking layer near the emission layer since the compound has a high T₁ energy (2.86 eV, 433 nm) and a deep LUMO level (2.7 eV or more).

In order to use the compound in a hole blocking layer of an organic light-emitting device, i.e., in a layer adjacent to an electron transport layer, the following point should be taken into consideration. That is, the compound has an adequate LUMO level with respect to the LUMO level of an electron transport material.

Representative examples of the electron transport material include tris(8-quinolinol)aluminum(III), 4,7-diphenyl-1,10-phenanthroline, and 2,9-dimethyl-4,7-diphenyl-1,10-phenanthroline. The LUMO levels of these electron transport materials are deep, i.e., 2.8 eV, 3.2 eV, and 3.3 eV, respectively.

Accordingly, the material used in the adjacent hole blocking layer needs to have an adequate LUMO level with respect to the LUMO level of the electron transport material. The LUMO level may be 2.7 eV or higher. At a LUMO level less than 2.7 eV, the difference (energy barrier) in LUMO level between the material used in the hole blocking layer and the electron transport material is large and the voltage for driving the light-emitting device is increased.

Since the LUMO level of the spiro(anthracene-9,9′-fluoren)-10-one compound of the invention is 2.7 eV or higher, the voltage for driving the light-emitting device does not increase much even when the compound is used in the hole blocking layer.

When the compound is used in the hole blocking layer of an organic light-emitting device, the following point should also be taken into consideration. That is, the compound has high electron mobility with respect to the hole mobility.

The spiro(anthracene-9,9′-fluoren)-10-one compound of this embodiment is a compound free of substituents having hole transport property, e.g., aryl amino and aryl carbazolyl groups. Accordingly, the electron transport property derived from the carbonyl group remains uninhibited and the electron mobility is high with respect to the hole mobility.

In order to use the spiro(anthracene-9,9′-fluoren)-10-one compound of the embodiment in an emission layer of an organic light-emitting device (an accessory component of a host material), the following point should be taken into consideration. That is, the compound has an adequate band gap with respect to the emission color of an emission material used in the organic light-emitting device.

The spiro(anthracene-9,9′-fluoren)-10-one compound of this embodiment has an aryl group, e.g., a biphenyl group, introduced into a site where conjugation with the anthrone skeleton is continued in order to narrow the band gap. Choices of the substitution sites are 1- to 8-positions of the formula below:

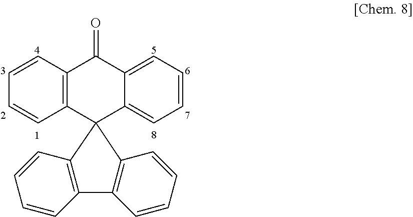 [SMILES: O=C1c2ccccc2C2(c3ccccc31)c1ccccc1-c1ccccc12]

Possible positions to which aryl groups are introduced are the 2-, 3-, 6-, and 7-positions. In this embodiment, aryl groups are introduced to one of 2-and 3-positions and one of 6- and 7-positions. With such substitution positions, the conjugation can be expanded and the band gap can be narrowed. Thus, substituents can be introduced to substitution positions that have less steric hindrance with the anthrone skeleton.

When a phosphorescent material is used as the emission material and the spiro(anthracene-9,9′-fluoren)-10-one compound of this embodiment is used in a hole blocking layer or as an accessory component of a host material of an emission layer, it is important that the T₁ energy of the compound satisfies a particular condition.

The T₁ energy of spiro(anthracene-9,9′-fluoren)-10-one which forms the basic skeleton (backbone) of the spiro(anthracene-9,9′-fluoren)-10-one compound of the embodiment is 433 nm. Since the backbone itself has a high T₁, various substituents can be introduced to decrease the T₁ energy in accordance to the emission spectrum of an emission material.

The T₁ energy of the spiro(anthracene-9,9′-fluoren)-10-one compound is also affected by the T₁ energy of the aryl group substituting one of the 2- and 3-positions and that of the aryl group substituting one of the 6- and 7-positions.

The T₁ energy (on a wavelength basis) of various aryls is presented in Table 1 below.

When the color of emission of the phosphorescent material is blue to green (maximum peak in the spectrum is in the range of 440 nm to 530 nm), aryls that have a higher T₁ energy are selected. Among the aryls in Table 1 below, benzene, benzothiophene, benzofuran, fluorene, triphenylene, biphenylene, terphenylene, phenanthrene, and naphthalene having T₁ energy of 500 nm or less are preferred, and benzene, benzothiophene, benzofuran, fluorene, triphenylene biphenylene, and terphenylene having T₁ energy of 450 nm or less are particularly preferable. When fluorene is used, dimethylfluorene is preferably used as shown by the structural formula of an example compound below.

TABLE 1

| | | T₁ energy |
|---|---|---|
| | | on a |
| | Structural | wavelength |
| Name | formulae | basis |
| Benzene | 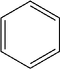 | 339 nm |
| Benzothiophene | 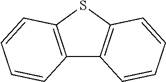 [SMILES: c1ccc2c(c1)sc1ccccc12] | 415 nm |
| Benzofuran | 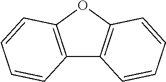 [SMILES: c1ccc2c(c1)oc1ccccc12] | 417 nm |
| Fluorene | 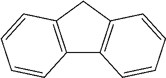 [SMILES: c1ccc2c(c1)Cc1ccccc1-2] | 422 nm |
| Triphenylene | 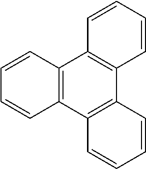 [SMILES: c1ccc2c(c1)c1ccccc1c1ccccc21] | 427 nm |
| Biphenylene | 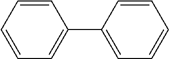 [SMILES: c1ccc(-c2ccccc2)cc1] | 436 nm |
| Terphenylene | 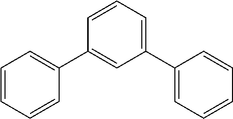 [SMILES: c1ccc(-c2cccc(-c3ccccc3)c2)cc1] | 445 nm |
| Phenanthrene | 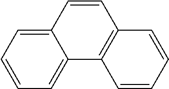 [SMILES: c1ccc2c(c1)ccc1ccccc12] | 459 nm |
| Naphthalene | 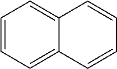 | 472 nm |
| Chrysene | 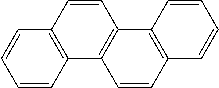 [SMILES: c1ccc2c(c1)ccc1c3ccccc3ccc21] | 500 nm |
| Pyrene | 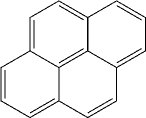 [SMILES: c1cc2ccc3cccc4ccc(c1)c2c34] | 589 nm |
| Anthracene | 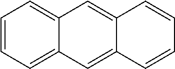 [SMILES: c1ccc2cc3ccccc3cc2c1] | 672 nm |

As described above, the compound of the embodiment has a deep LUMO level (2.7 eV or more), high electron mobility, and high T₁ energy. Thus, when the compound is used as a material for a hole blocking layer, the driving voltage of the device can be lowered while achieving high efficiency.

The compound of the embodiment also has a narrow band bap and high T₁ energy. Thus, when the compound is used as a host material of an emission layer, the driving voltage of the device can be lowered while achieving high efficiency.

In all cases, the compound contributes to decreasing the driving voltage of the device and electrochemical load imposed on the device. Thus, the lifetime of the device can be extended.

Examples of the spiro(anthracene-9,9′-fluoren)-10-one Compound of the Embodiment

Examples of the specific structural formulae of the spiro(anthracene-9,9′-fluoren)-10-one compound are as follows.

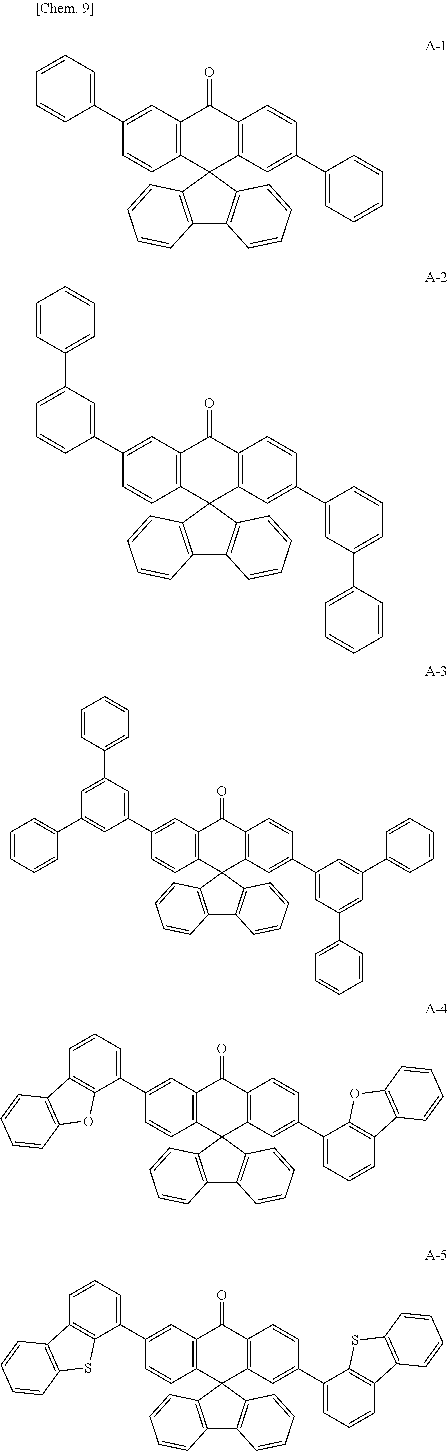 [SMILES: O=C1c2cc(-c3ccccc3)ccc2C2(c3cc(-c4ccccc4)ccc31)c1ccccc1-c1ccccc12 ; O=C1c2cc(-c3cccc(-c4ccccc4)c3)ccc2C2(c3cc(-c4cccc(-c5ccccc5)c4)ccc31)c1ccccc1-c1ccccc12 ; O=C1c2cc(-c3cc(-c4ccccc4)cc(-c4ccccc4)c3)ccc2C2(c3cc(-c4cc(-c5ccccc5)cc(-c5ccccc5)c4)ccc31)c1ccccc1-c1ccccc12 ; O=C1c2cc(-c3cccc4c3oc3ccccc34)ccc2C2(c3cc(-c4cccc5c4oc4ccccc45)ccc31)c1ccccc1-c1ccccc12 ; O=C1c2cc(-c3cccc4c3sc3ccccc34)ccc2C2(c3cc(-c4cccc5c4sc4ccccc45)ccc31)c1ccccc1-c1ccccc12]

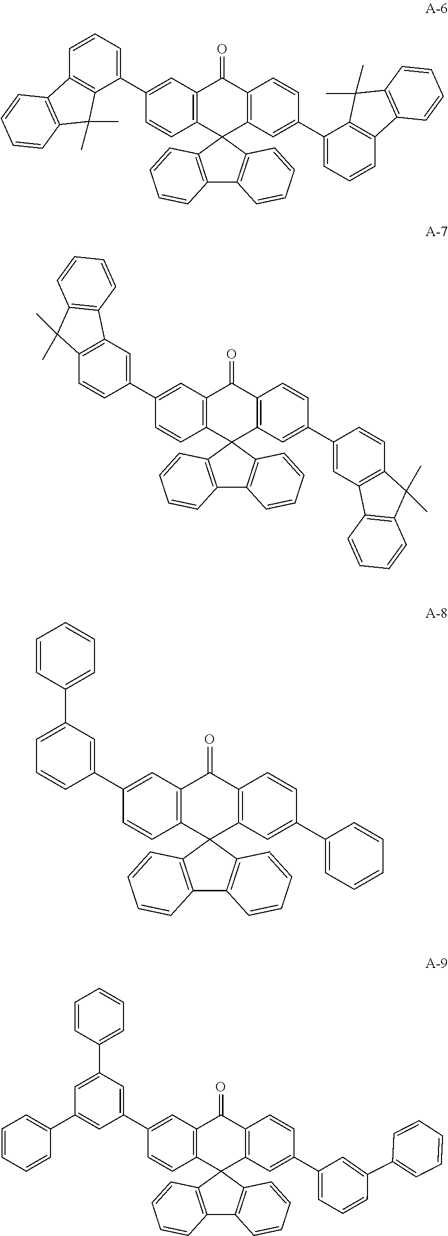

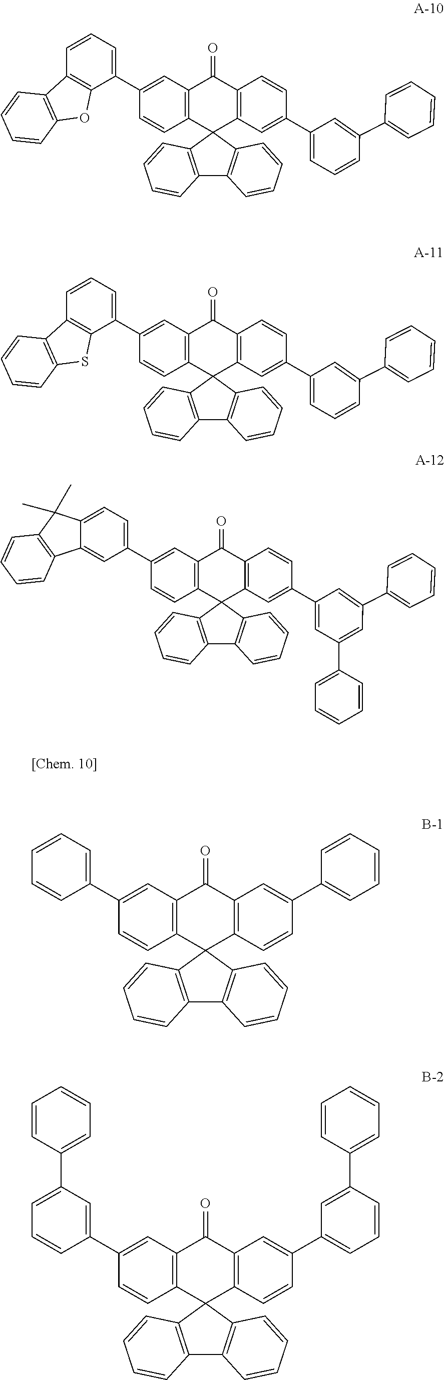 [SMILES: O=C1c2cc(-c3cccc4c3oc3ccccc34)ccc2C2(c3cc(-c4cccc(-c5ccccc5)c4)ccc31)c1ccccc1-c1ccccc12 ; O=C1c2cc(-c3cccc4c3sc3ccccc34)ccc2C2(c3cc(-c4cccc(-c5ccccc5)c4)ccc31)c1ccccc1-c1ccccc12 ; CC1(C)c2ccccc2-c2cc(-c3ccc4c(c3)C(=O)c3ccc(-c5cc(-c6ccccc6)cc(-c6ccccc6)c5)cc3C43c4ccccc4-c4ccccc43)ccc21 ; O=C1c2cc(-c3ccccc3)ccc2C2(c3ccc(-c4ccccc4)cc31)c1ccccc1-c1ccccc12 ; O=C1c2cc(-c3cccc(-c4ccccc4)c3)ccc2C2(c3ccc(-c4cccc(-c5ccccc5)c4)cc31)c1ccccc1-c1ccccc12]

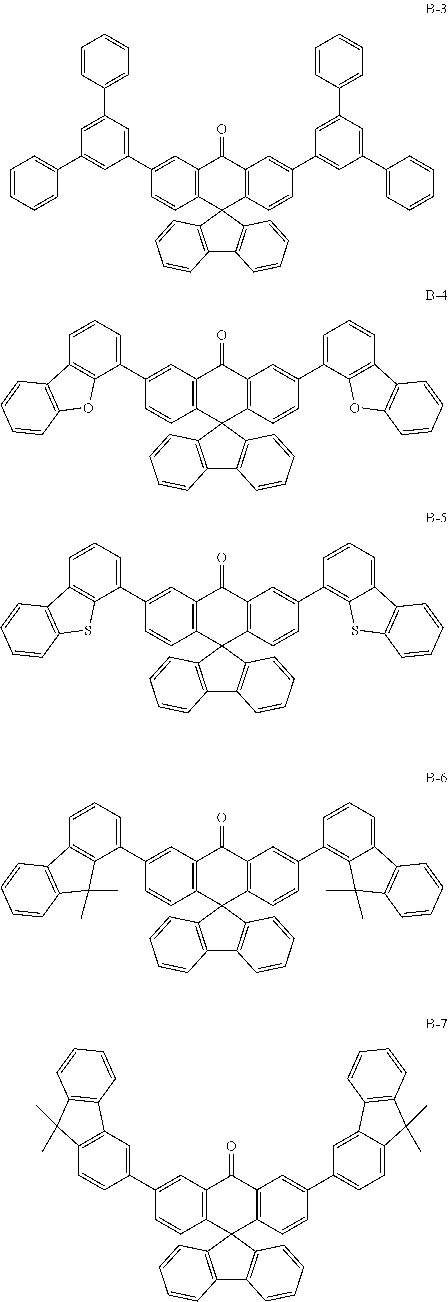

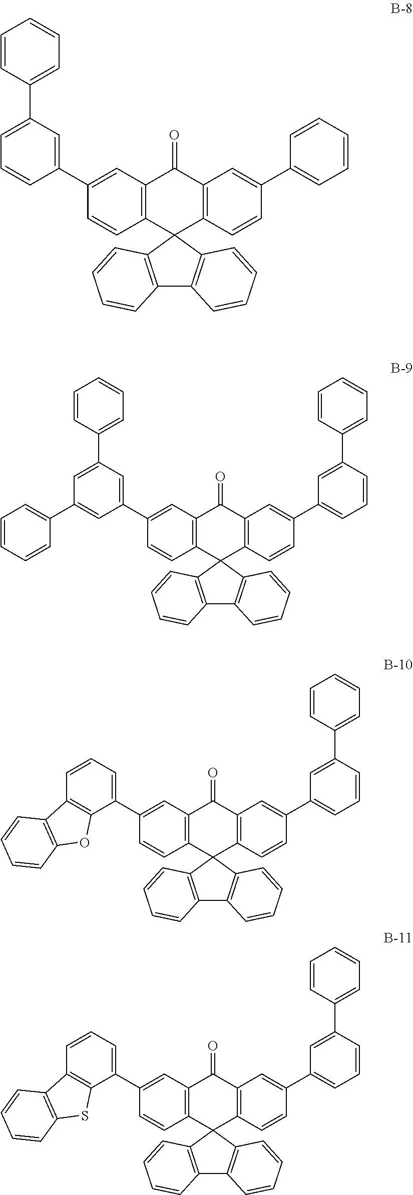 [SMILES: O=C1c2cc(-c3ccccc3)ccc2C2(c3ccc(-c4cccc(-c5ccccc5)c4)cc31)c1ccccc1-c1ccccc12 ; O=C1c2cc(-c3cccc(-c4ccccc4)c3)ccc2C2(c3ccc(-c4cc(-c5ccccc5)cc(-c5ccccc5)c4)cc31)c1ccccc1-c1ccccc12 ; O=C1c2cc(-c3cccc(-c4ccccc4)c3)ccc2C2(c3ccc(-c4cccc5c4oc4ccccc45)cc31)c1ccccc1-c1ccccc12 ; O=C1c2cc(-c3cccc(-c4ccccc4)c3)ccc2C2(c3ccc(-c4cccc5c4sc4ccccc45)cc31)c1ccccc1-c1ccccc12]

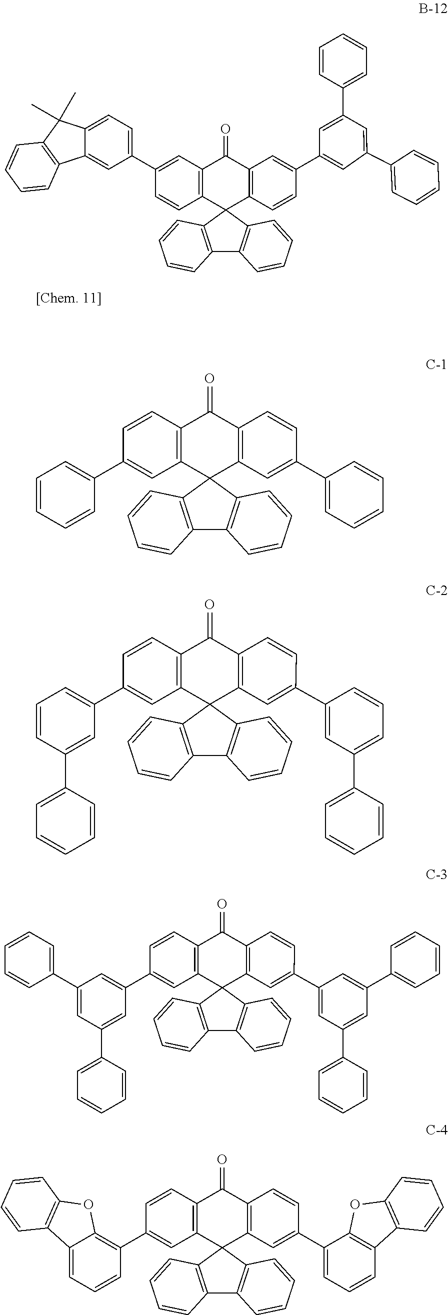 [SMILES: CC1(C)c2ccccc2-c2cc(-c3ccc4c(c3)C(=O)c3cc(-c5cc(-c6ccccc6)cc(-c6ccccc6)c5)ccc3C43c4ccccc4-c4ccccc43)ccc21 ; O=C1c2ccc(-c3ccccc3)cc2C2(c3cc(-c4ccccc4)ccc31)c1ccccc1-c1ccccc12 ; O=C1c2ccc(-c3cccc(-c4ccccc4)c3)cc2C2(c3cc(-c4cccc(-c5ccccc5)c4)ccc31)c1ccccc1-c1ccccc12 ; O=C1c2ccc(-c3cc(-c4ccccc4)cc(-c4ccccc4)c3)cc2C2(c3cc(-c4cc(-c5ccccc5)cc(-c5ccccc5)c4)ccc31)c1ccccc1-c1ccccc12 ; O=C1c2ccc(-c3cccc4c3oc3ccccc34)cc2C2(c3cc(-c4cccc5c4oc4ccccc45)ccc31)c1ccccc1-c1ccccc12]

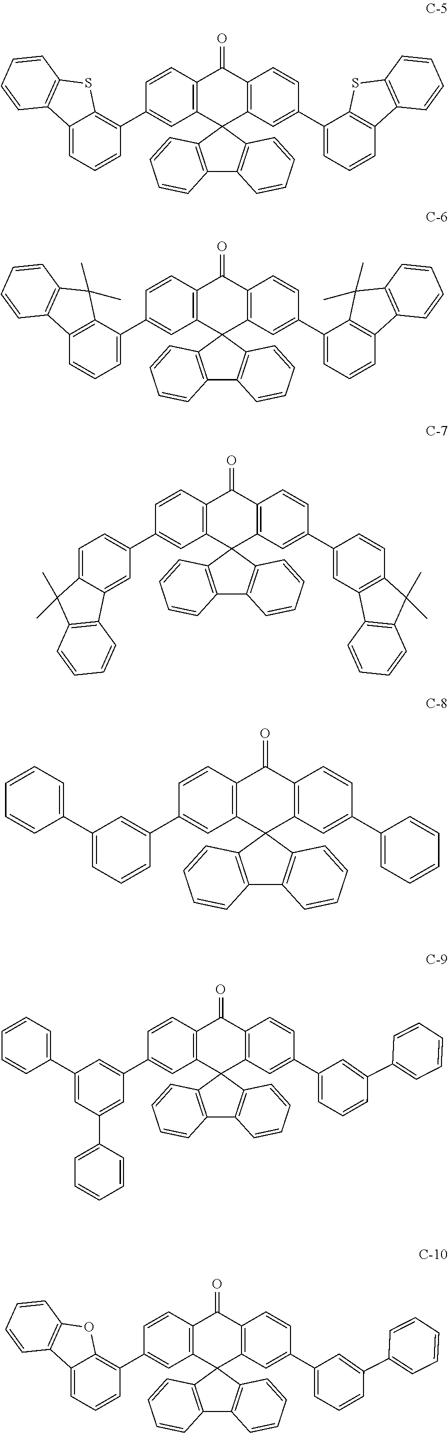

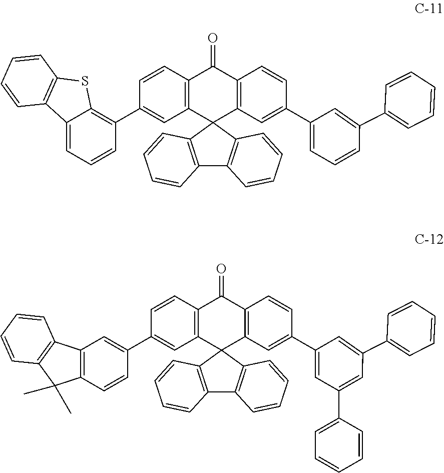 [SMILES: O=C1c2ccc(-c3cccc(-c4ccccc4)c3)cc2C2(c3cc(-c4cccc5c4sc4ccccc45)ccc31)c1ccccc1-c1ccccc12 ; CC1(C)c2ccccc2-c2cc(-c3ccc4c(c3)C3(c5cc(-c6cc(-c7ccccc7)cc(-c7ccccc7)c6)ccc5C4=O)c4ccccc4-c4ccccc43)ccc21]

Compounds of Group A are compounds represented by general formula [1] having substituents at Ar₁ and Ar₃ (or Ar₂ and Ar₄). Of the two substituents, one is substituted at a para (p) position with respect to the carbonyl in the anthrone skeleton, in other words, at a position where the conjugation expands. Thus, the electron transport property can be improved.

Moreover, since Group A compounds are asymmetric compounds having Ar₃ (Ar₄) at a position asymmetric to Ar₁ (Ar₂), a highly stable amorphous film can be obtained since crystallization is suppressed during manufacture of a thin film.

Compounds of Group B are compounds represented by general formula [1] having substituents at Ar₁ and Ar₄. Since the two substituents are at meta (m) positions with respect to the carbonyl in the anthrone skeleton, i.e., positions that narrow the conjugation compared to the para positions described above, a compound having higher T₁ energy can be obtained.

Compounds of Group C are compounds represented by general formula [1] having substituents at Ar₂ and Ar₃. Since the two substituents are at para (p) positions with respect to the carbonyl in the anthrone skeleton, i.e., positions that expand the conjugation, a compound having high electron transport property can be obtained.

In this embodiment, selection may be freely made from compounds of Groups A to C. When the compound is to be used in a single-layer film as an electron transport material, film stability is also needed. Thus, compounds of Group A are preferably used. When the compound is used as an assisting material for the emission layer, the assisting material must have high T₁ energy as the emission color approaches blue. Thus, selection may be made from the compounds of Group B.

The two substituents of the spiro(anthracene-9,9′-fluoren)-10-one compound of the embodiment may be the same aryl group or different aryl groups. A compound having T₁ energy of 2.3 eV or more and a LUMO level of 2.7 eV or more can be obtained even when the two substituents are different.

Method for Synthesizing the spiro(anthracene-9,9′-fluoren)-10-one Compound

A method for synthesizing the spiro(anthracene-9,9′-fluoren)-10-one compound will now be described.

A dihalide of the raw material, spiro(anthracene-9,9′-fluoren)-10-one can be synthesized through the scheme below, in which compounds [3], [7a], and [7b] are dihalides. A compound [1] can be purchased from Tokyo Chemical Industry Co., Ltd. (reactant code: No. D3182, trade name: dibromoanthraquinone). The synthetic method for a compound [4] is described in Journal of Organometallic Chemistry (1977), 128 (1), pp. 95-98.

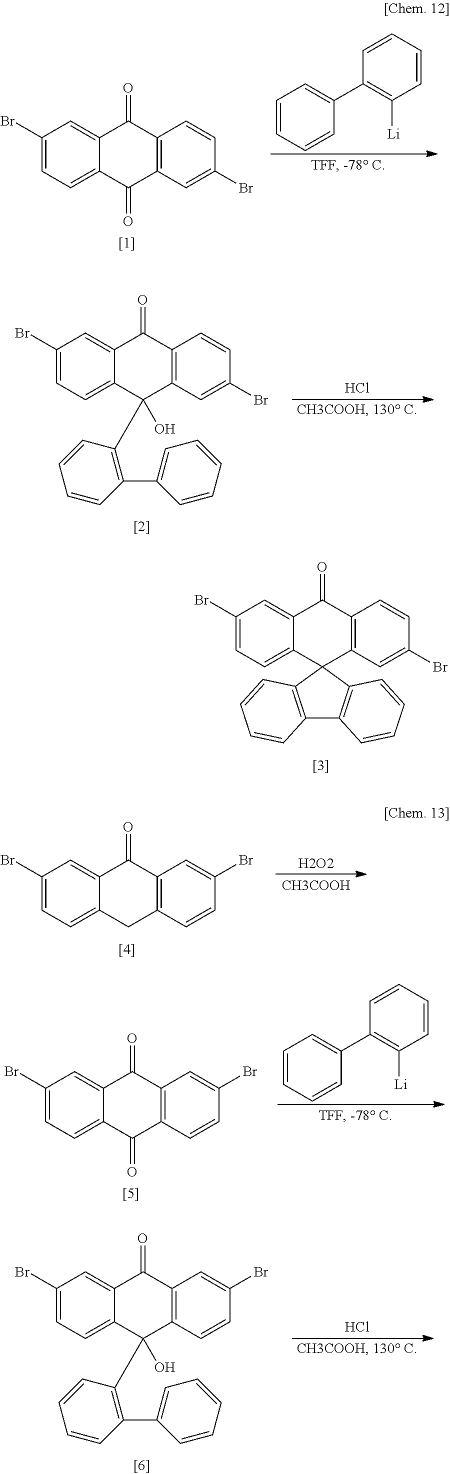

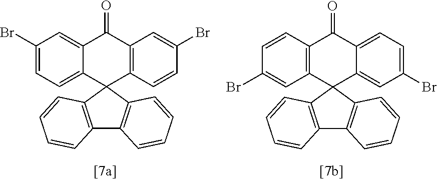 [SMILES: O=C1c2cc(Br)ccc2C2(c3ccc(Br)cc31)c1ccccc1-c1ccccc12 ; O=C1c2ccc(Br)cc2C2(c3cc(Br)ccc31)c1ccccc1-c1ccccc12]

The spiro(anthracene-9,9′-fluoren)-10-one compound of this embodiment can also be synthesized through a coupling reaction between the raw material, dihalide described above and boronic acid or a borate compound of aryl in the presence of a Pd catalyst, as illustrated in the schemes below.

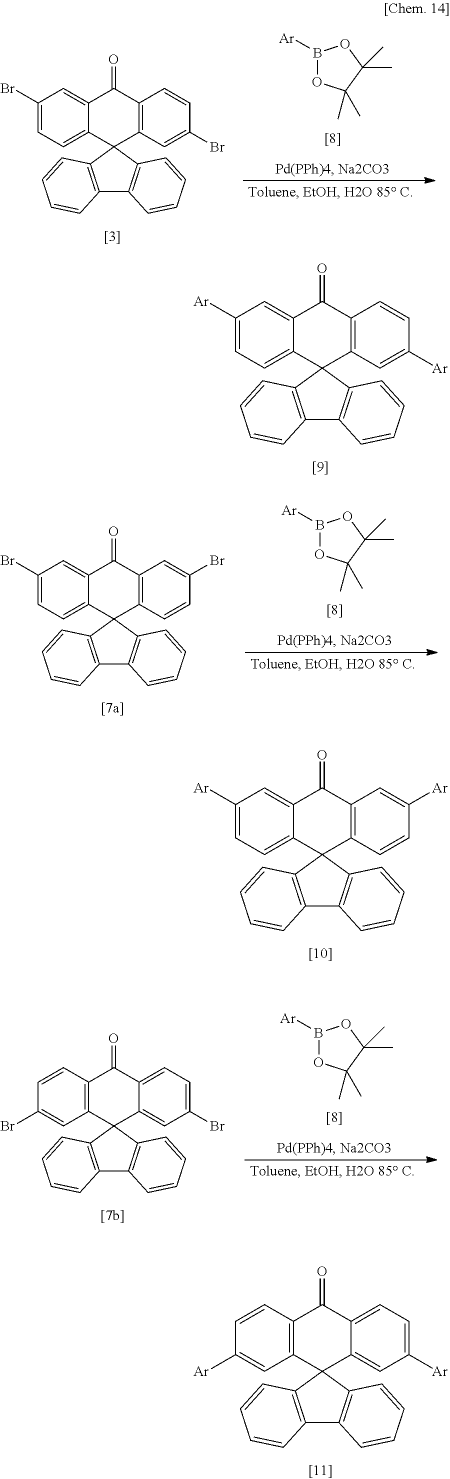

In [9], [10], and [11], the aryl groups (Ar) are each individually selected from a phenyl group, a biphenyl group, a terphenyl group, a fluorenyl group, a triphenylene group, a dibenzofuran group, and a dibenzothiophene group.

When the spiro(anthracene-9,9′-fluoren)-10-one compound is used in an organic light-emitting device, sublimation purification may be conducted as the last purification before fabrication of the device. This is because sublimation purification yields a high purification effect in increasing the purity of an organic compound. In general, sublimation purification requires a high temperature as the molecular weight of the organic compound increases, and pyrolysis tends to occur at such a high temperature. Accordingly, the organic compound used in the organic light-emitting device may have a molecular weight of 1000 or less so that sublimation purification can be conducted without excessive heating.

### Light-emitting Device

An organic light-emitting device according to an embodiment of the present invention will now be described.

The organic light-emitting device includes a pair of electrodes opposing each other, i.e., an anode and a cathode, and an organic compound layer interposed between the electrodes. The organic compound layer of the organic light-emitting device contains a spiro(anthracene-9,9′-fluoren)-10-one compound represented by general formula [1].

Examples of the structure that can be employed in the organic light-emitting device of this embodiment includes an anode/emission layer/cathode structure, an anode/hole transport layer/electron transport layer/cathode structure, an anode/hole transport layer/emission layer/electron transport layer/cathode structure, an anode/hole injection layer/hole transport layer/emission layer/electron transport layer/cathode structure, and an anode/hole transport layer/emission layer/hole blocking layer/electron transport layer/cathode structure, the layers in the structures being sequentially formed on a substrate. Note that these five types of multilayer organic light-emitting devices are only basic device structures and the structure of the organic light-emitting device that uses the compound of the embodiment is not limited to these. For example, an insulating layer may be formed between an electrode and an organic compound layer, an adhesive layer or an interference layer may be provided in addition, and the electron transport layer or hole transport layer may be constituted by two layers having different ionization potentials.

The device may be of a top-emission type in which light is output from the substrate-side electrode or of a bottom-emission type in which light is output from the side remote from the substrate, or may be configured to output light from both sides.

The spiro(anthracene-9,9′-fluoren)-10-one compound of the embodiment can be used in an organic compound layer of an organic light-emitting device having any layer structure. For example, the compound is preferably used in the electron transport layer, the hole blocking layer, or the emission layer, and more preferably used in the hole blocking layer or the emission layer. When the compound is used in the emission layer, the compound is preferably used as an accessory component (second host material or host material 2) of the host material. In this case, the main component of the host material is called a “first host material” or “host material 1”.

The emission layer may contain a host material and a guest material (also referred to as “emission material”). A host material is a material other than the guest material.

The emission layer may contain two or more host materials. The concentration of the phosphorescent material is 0.01 wt % to 50 wt % and preferably 0.1 wt % to 20 wt % relative to the total amount of the materials constituting the emission layer. The concentration is more preferably 10 wt % or less to prevent concentration quenching. The emission material may be homogeneously contained in all parts of the layer composed of the host materials, may be contained in the layer by having a concentration gradient, or may be contained in some parts of the layer, leaving other parts of the layer solely composed of host materials and thus free of the emission material.

When a phosphorescent material is used as the guest material, the phosphorescent material may be a metal complex such as an iridium complex, a platinum complex, a rhenium complex, a copper complex, an europium complex, or a ruthenium complex. Of these, an iridium complex having a high phosphorescent property is preferred. The emission layer may contain two or more phosphorescent materials so that transmission of excitons and carriers can be assisted.

The emission color of the phosphorescent material is not particularly limited but is preferably blue to green with a maximum emission peak wavelength in the range of 440 nm to 530 nm.

In general, the T₁ energy of a host material must be higher than the T₁ energy of a phosphorescent material to prevent a decrease in emission efficiency caused by nonradiative deactivation.

The spiro(anthracene-9,9′-fluoren)-10-one compound of this embodiment has a spiro(anthracene-9,9′-fluoren)-10-one basic skeleton (backbone) having T₁ energy of 433 nm. This T₁ energy is higher than that of a blue phosphorescent material. Accordingly, when the spiro(anthracene-9,9′-fluoren)-10-one compound is used in an emission layer of a blue to green organic light-emitting device, high emission efficiency can be achieved.

Specific examples of the iridium complex used as a phosphorescent material are as follows. These examples do not limit the scope of the present invention.

Examples of the iridium complex are as follows.

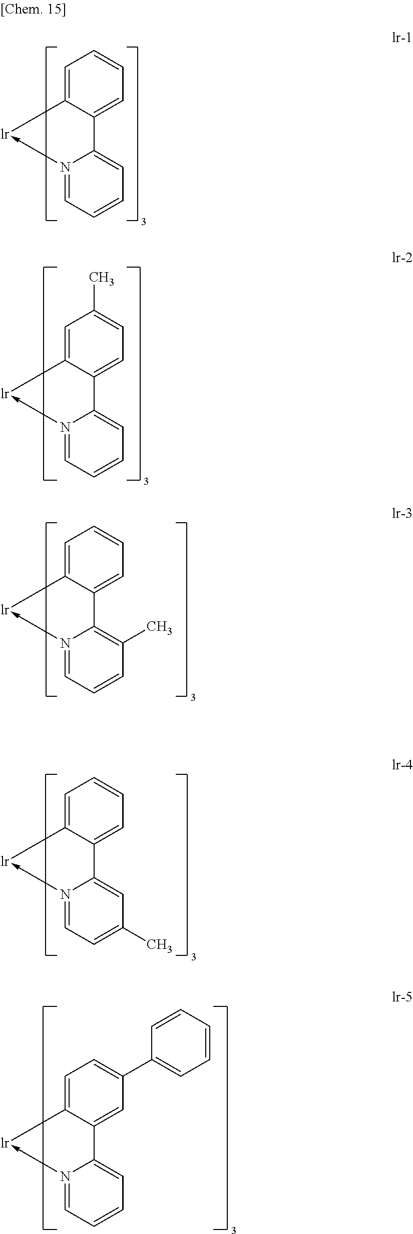

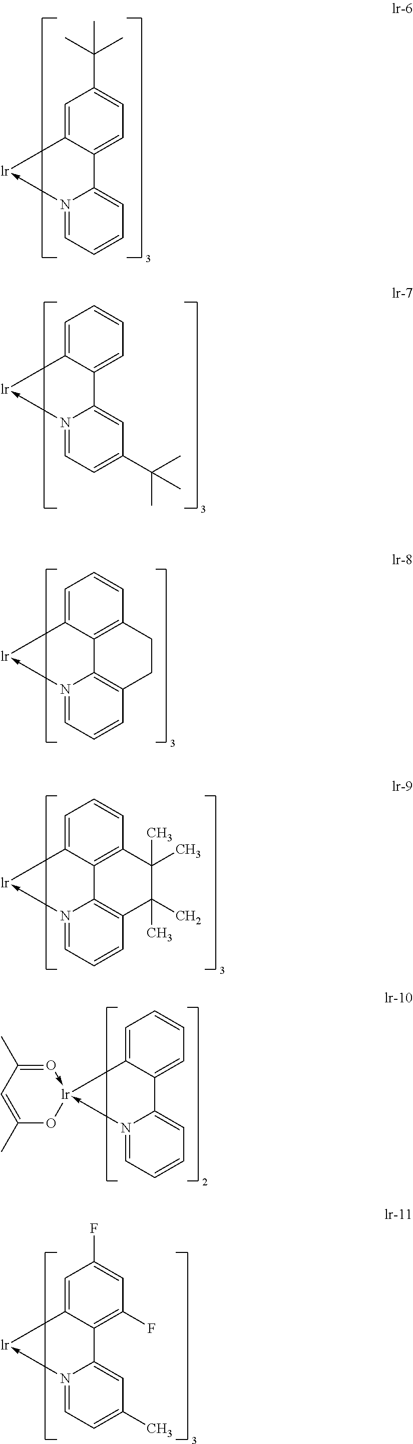

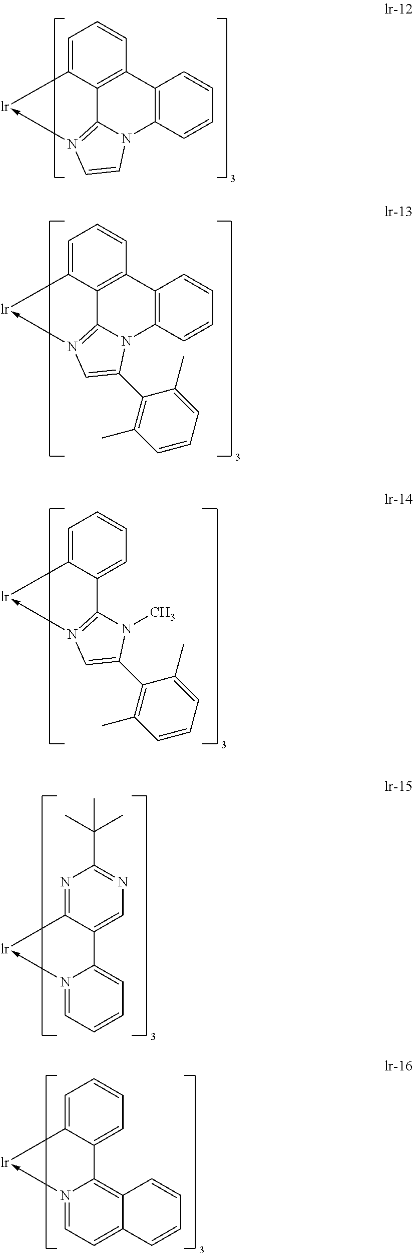

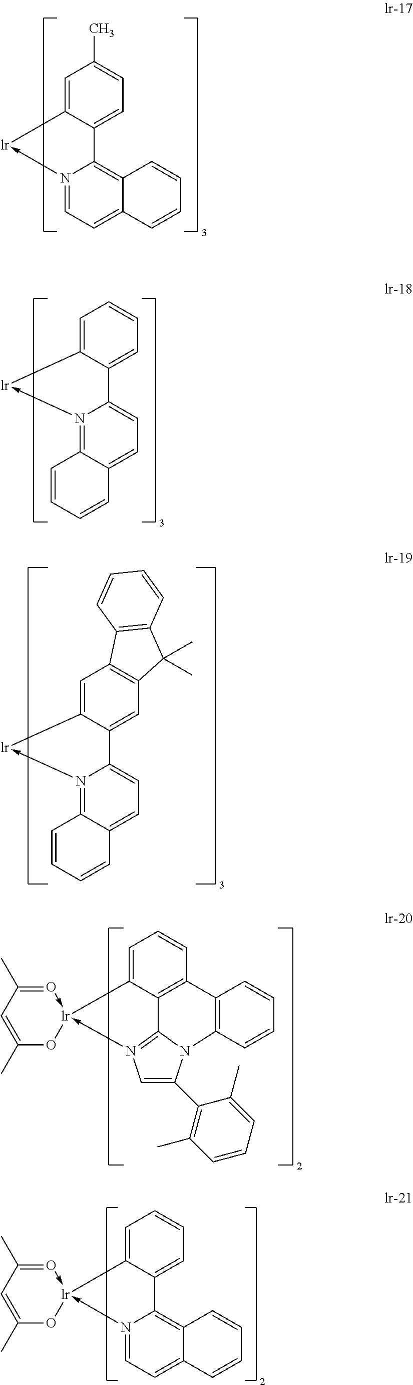

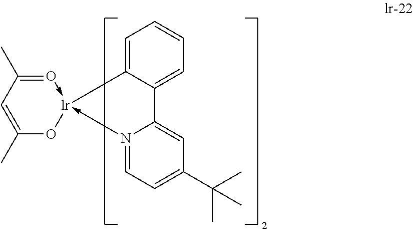

Examples of the host material are as follows.

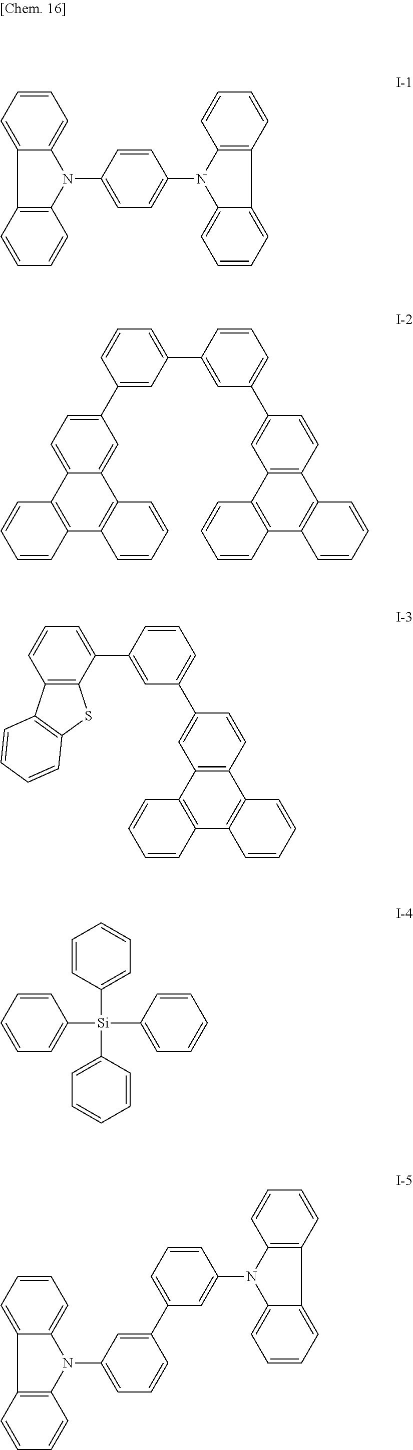

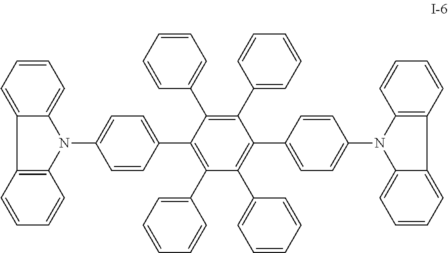 [SMILES: c1ccc(-c2c(-c3ccccc3)c(-c3ccc(-n4c5ccccc5c5ccccc54)cc3)c(-c3ccccc3)c(-c3ccccc3)c2-c2ccc(-n3c4ccccc4c4ccccc43)cc2)cc1]

If needed, low-molecular-weight and high-molecular weight compounds of related art can be used in addition to the spiro(anthracene-9,9-fluoren)-10-one compound. In particular, a hole injection compound, a hole transport compound, a host material, a light-emitting compound, an electron injection compound, an electron transport compound, or the like may be used in combination.

The hole injection/transport material preferably has high hole mobility so that the hole can be easily injected from the anode and the injected holes can be transported to the emission layer. Examples of the high-molecular-weight and low-molecular-weight compounds having hole injection/transport property include triarylamine derivatives, phenylenediamine derivatives, stilbene derivatives, phthalocyanine derivatives, porphyrin derivatives, poly(vinyl carbazole), poly(thiophene), and other conductive polymers.

Examples of the emission material contributing mainly to a light-emitting function include phosphorescent guest materials described above and derivatives thereof, fused ring compounds (e.g., fluorene derivatives, naphthalene derivatives, pyrene derivatives, perylene derivatives, tetracene derivatives, anthracene derivatives, and rubrene), quinacridone derivatives, coumarin derivatives, stilbene derivatives, organic aluminum complexes such as tris(8-quinolinolato)aluminum, organic beryllium complexes, and polymer derivatives such as poly(phenylene vinylene) derivatives, poly(fluorene) derivatives, and poly(phenylene) derivatives.

The electron injection/transport material can be freely selected from those materials into which electrons can be easily injected from the cathode and in which injected electrons can be transported to the emission layer. The selection is made by considering the balance with the hole mobility of the hole injection/transport material, etc. Examples of the material having electron injection/transport property include oxadiazole derivatives, oxazole derivatives, pyrazine derivatives, triazole derivatives, triazine derivatives, quinoline derivatives, quinoxaline derivatives, phenanthroline derivatives, and organic aluminum complexes.

The anode material may have a large work function. Examples of the anode material include single metals such as gold, platinum, silver, copper, nickel, palladium, cobalt, selenium, vanadium, and tungsten or alloys thereof, and metal oxides such as tin oxide, zinc oxide, indium oxide, indium tin oxide (ITO), and indium zinc oxide. Conductive polymers such as polyaniline, polypyrrole, and polythiophene may also be used. These anode materials may be used alone or in combination. The anode may be constituted by one layer or two or more layers.

The cathode material may have a small work function. Examples of the cathode material include alkali metals such as lithium, alkaline earth metals such as calcium, and single metals such as aluminum, titanium, manganese, silver, lead, and chromium. The single metals may be combined and used as alloys. For example, magnesium-silver, aluminum-lithium, and aluminum-magnesium alloys and the like can be used. Metal oxides such as indium tin oxide (ITO) can also be used. These cathode materials may be used alone or in combination. The cathode may be constituted by one layer or two or more layers.

A layer containing the organic compound of the embodiment and a layer composed of other organic compound of the organic light-emitting device of the embodiment are prepared by the methods below. Typically, thin films are formed by vacuum vapor deposition, ionization deposition, sputtering, plasma, or coating using an adequate solvent (spin-coating, dipping, casting, a Langmuir Blodgett method, and an ink jet method). When layers are formed by vacuum vapor deposition or a solution coating method, crystallization is suppressed and stability over time can be improved. When a coating method is employed, an adequate binder resin may be additionally used to form a film.

Examples of the binder resin include, but are not limited to, polyvinylcarbazole resins, polycarbonate resins, polyester resins, ABS resins, acrylic resins, polyimide resins, phenolic resins, epoxy resins, silicone resins, and urea resins. These binder resins may be used alone as a homopolymer or in combination of two or more as a copolymer. If needed, known additives such as a plasticizer, an antioxidant, and an ultraviolet absorber may be used in combination.

### Usage of Organic Light-emitting Device

The organic light-emitting device of the embodiment may be used in a display apparatus or a lighting apparatus. The organic light-emitting device can also be used as exposure light sources of image-forming apparatuses and backlights of liquid crystal display apparatuses.

A display apparatus includes a display unit that includes the organic light-emitting device of this embodiment. The display unit has pixels and each pixel includes the organic light-emitting device of this embodiment. The display apparatus may be used as an image display apparatus of a personal computer, etc.

The display apparatus may be used in a display unit of an imaging apparatus such as digital cameras and digital video cameras. An imaging apparatus includes the display unit and an imaging unit having an imaging optical system for capturing images.

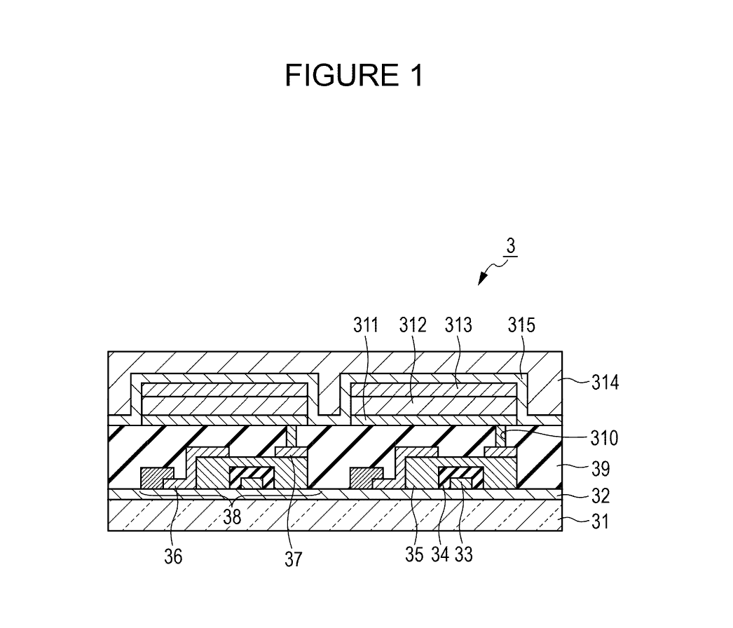

FIG. 1 is a schematic cross-sectional view of an image display apparatus having an organic light-emitting device in a pixel unit. In the drawing, two organic light-emitting devices and two thin film transistors (TFTs) are illustrated. One organic light-emitting device is connected to one TFT.

Referring to FIG. 1, in an image display apparatus 3, a moisture proof film 32 is disposed on a substrate 31 composed of glass or the like to protect components (TFT or organic layer) formed thereon. The moisture proof film 32 is composed of silicon oxide or a composite of silicon oxide and silicon nitride. A gate electrode 33 is provided on the moisture proof film 32. The gate electrode 33 is formed by depositing a metal such as Cr by sputtering.

A gate insulating film 34 covers the gate electrode 33. The gate insulating film 34 is obtained by forming a layer of silicon oxide or the like by a plasma chemical vapor deposition (CVD) method or a catalytic chemical vapor deposition (cat-CVD) method and patterning the film. A semiconductor layer 35 is formed over the gate insulating film 34 in each region that forms a TFT by patterning. The semiconductor layer 35 is obtained by forming a silicon film by a plasma CVD method or the like (optionally annealing at a temperature 290° C. or higher, for example) and patterning the resulting film according to the circuit layout.

A drain electrode 36 and a source electrode 37 are formed on each semiconductor layer 35. In sum, a TFT 38 includes a gate electrode 33, a gate insulating layer 34, a semiconductor layer 35, a drain electrode 36, and a source electrode 37. An insulating film 39 is formed over the TFT 38. A contact hole (through hole) 310 is formed in the insulating film 39 to connect between a metal anode 311 of the organic light-emitting device and the source electrode 37.

A single-layer or a multilayer organic layer 312 that includes an emission layer and a cathode 313 are stacked on the anode 311 in that order to constitute an organic light-emitting device that functions as a pixel. First and second protective layers 314 and 315 may be provided to prevent deterioration of the organic light-emitting device.

The switching device is not particularly limited and a metal-insulator-metal (MIM) element may be used instead of the TFT described above.

### EXAMPLES

### Example 1

### Synthesis of Example Compound A-2

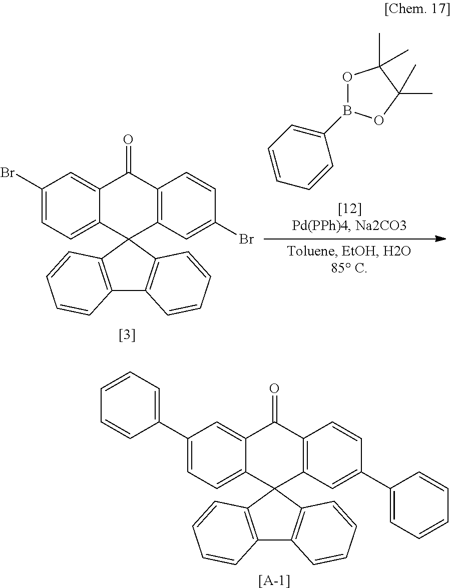

The following reagents and solvents were placed in a 200 mL round-bottomed flask.
[3]: 1 g (2 mmol)
[12] (phenylboronic acid): 0.8 g (4 mmol)
Pd(PPh)4 (tetrakis(triphenylphosphine)palladium(0)): 0.23 g (0.2 mmol)
Toluene: 50 mL
Ethanol: 20 mL
30 wt % Aqueous sodium carbonate solution: 30 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water, ethanol, and acetone to obtain a crude product. The crude product was dissolved in toluene under heating, subjected to hot filtration, and recrystallized twice with a toluene solvent. The obtained crystals were vacuum dried at 100° C. and purified by sublimation at 10⁻⁴ Pa and 300° C. As a result, 0.46 g (yield: 46%) of high-purity Example Compound A-1 was obtained.

The compound obtained was identified by mass spectroscopy.
Matrix-assisted laser desorption ionization-time-of-flight mass spectroscopy (MALDI-TOF-MS)
Observed value: m/z=496.6
Calculated value: C₂₈H₂₂O=496.2

The T₁ energy of Example Compound A-1 was measured by the following process.

A phosphorescence spectrum of a toluene diluted solution (about 10⁻⁴ mol/L) of Example Compound A-1 was measured in an Ar atmosphere at 77 K and an excitation wavelength of 310 nm. The T₁ energy was calculated from the peak wavelength of the first emission peak of the obtained phosphorescence spectrum. The T₁ energy was 460 nm on a wavelength basis.

The energy gap of Example Compound A-1 was measured by the following process.

Example Compound A-1 was vapor-deposited by heating on a glass substrate to obtain a deposited thin film 20 nm in thickness. An absorption spectrum of the deposited thin film was taken with an ultraviolet-visible spectrophotometer (V-560 produced by JASCO Corporation). The energy gap of Example Compound A-1 determined from the absorption edge of the absorption spectrum was 3.5 eV.

### Example 2

### Synthesis of Example Compound A-3

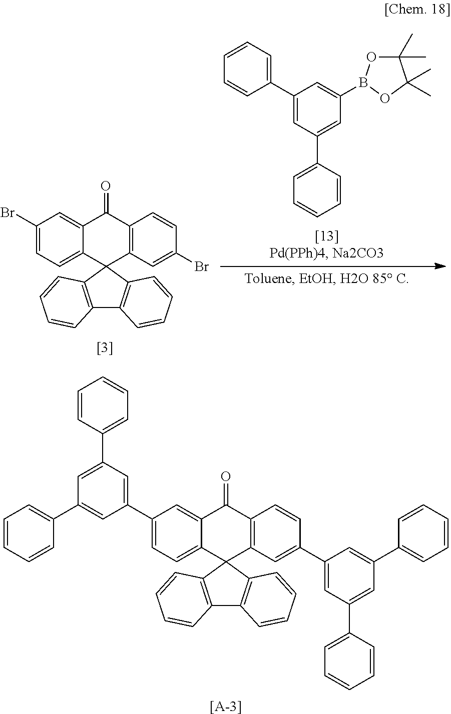

The following reagents and solvents were placed in a 200 mL round-bottomed flask.
[3]: 1 g (2 mmol)
[13] (terphenylboronic acid): 1.4 g (4 mmol)
Pd(PPh)4 (tetrakis(triphenylphosphine)palladium(0)): 0.23 g (0.2 mmol)
Toluene: 50 mL
Ethanol: 20 mL
30 wt % Aqueous sodium carbonate solution: 30 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water, ethanol, and acetone to obtain a crude product. The crude product was dissolved in toluene under heating, subjected to hot filtration, and recrystallized twice with a toluene solvent. The obtained crystals were vacuum dried at 100° C. and purified by sublimation at 10⁻⁴ Pa and 320° C. As a result, 0.33 g (yield: 21%) of high-purity Example Compound A-3 was obtained.

The compound obtained was identified by mass spectroscopy.
Matrix-assisted laser desorption ionization-time-of-flight mass spectroscopy (MALDI-TOF-MS)
Observed value: m/z=801.0
Calculated value: C₂₈H₂₂O=800.3
The T₁ energy of Example Compound A-3 measured as in Example 1 was 471 nm on a wavelength basis.

The energy gap of Example Compound A-3 determined as in Example 1 was 3.4 eV.

### Example 3

### Synthesis of Example Compound A-7

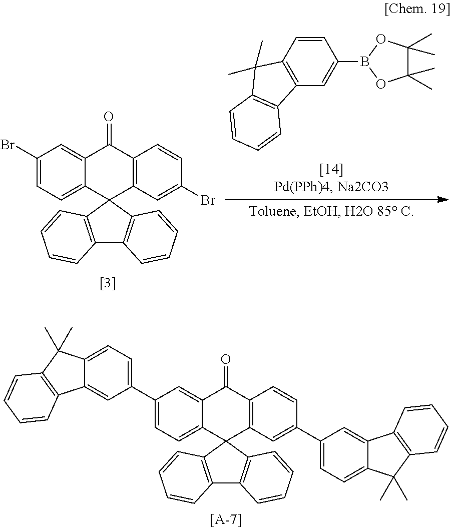

The following reagents and solvents were placed in a 200 mL round-bottomed flask.
[3]: 1 g (2 mmol)
[14] (fluorenylboronic acid: 1.3 g (4 mmol)
Pd(PPh)4 (tetrakis(triphenylphosphine)palladium(0)): 0.23 g (0.2 mmol)
Toluene: 50 mL
Ethanol: 20 mL
30 wt % Aqueous sodium carbonate solution: 30 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water, ethanol, and acetone to obtain a crude product. The crude product was dissolved in toluene under heating, subjected to hot filtration, and recrystallized twice with a toluene solvent. The obtained crystals were vacuum dried at 100° C. and purified by sublimation at 10⁻⁴ Pa and 315° C. As a result, 0.39 g (yield: 27%) of high-purity Example Compound A-7 was obtained.

### [MALDI-TOF-MS]

Observed value: m/z=728.9
Calculated value: 728.3
The T₁ energy of Example Compound A-7 measured as in Example 1 was 480 nm on a wavelength basis.

The energy gap of Example Compound A-7 determined as in Example 1 was 3.2 eV.

### Example 4

### Synthesis of Example Compound B-1

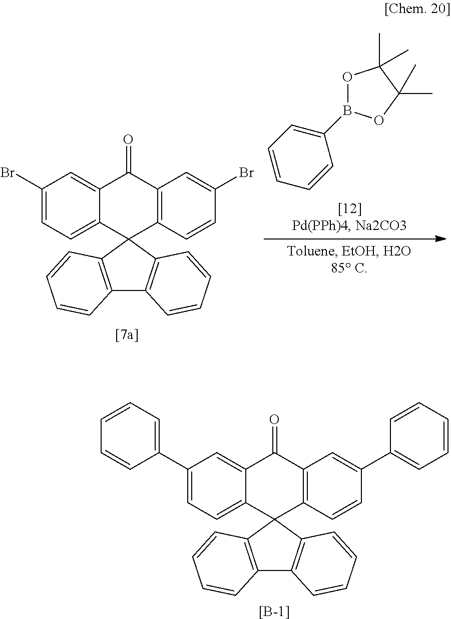

The following reagents and solvents were placed in a 200 mL round-bottomed flask.
[7a]: 1 g (2 mmol)
[12] (phenylboronic acid): 0.8 g (4 mmol)
Pd(PPh)4 (tetrakis(triphenylphosphine)palladium(0)): 0.23 g (0.2 mmol)
Toluene: 50 mL
Ethanol: 20 mL
30 wt % Aqueous sodium carbonate solution: 30 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water, ethanol, and acetone to obtain a crude product. The crude product was dissolved in toluene under heating, subjected to hot filtration, and recrystallized twice with a toluene solvent. The obtained crystals were vacuum dried at 100° C. and purified by sublimation at 10⁻⁴ Pa and 300° C. As a result, 0.55 g (yield: 56%) of high-purity Example Compound B-1 was obtained.

The obtained compound was identified by mass spectroscopy.

### [MALDI-TOF-MS]

Observed value: m/z=496.7
Calculated value: C₂₈H₂₂O=496.2
The T₁ energy of Example Compound B-1 measured as in Example 1 was 454 nm on a wavelength basis.

The energy gap of Example Compound B-1 measured as in Example 1 was 3.7 eV.

### Example 5

### Synthesis of Example Compound B-3

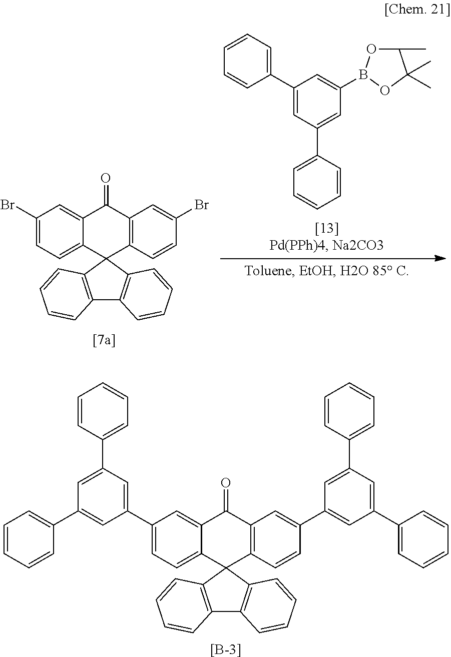

The following reagents and solvents were placed in a 200 mL round-bottomed flask.
[7a]: 1 g (2 mmol)
[13] (terphenylboronic acid): 1.4 g (4 mmol)
Pd(PPh)4 ((tetrakis(triphenylphosphine)palladium(0)): 0.23 g (0.2 mmol)
Toluene: 50 mL
Ethanol: 20 mL
30 wt % Aqueous sodium carbonate solution: 30 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water, ethanol, and acetone to obtain a crude product. The crude product was dissolved in chlorobenzene under heating, subjected to hot filtration, and recrystallized twice with a chlorobenzene solvent. The obtained crystals were vacuum dried at 100° C. and purified by sublimation at 10⁻⁴ Pa and 340° C. As a result, 0.51 g (yield: 32%) of high-purity Example Compound B-3 was obtained.

### [MALDI-TOF-MS]

Observed value: m/z=800.9
Calculated value: 800.3
The T₁ energy of Example Compound B-3 measured as in Example 1 was 461 nm on a wavelength basis.

The energy gap of Example Compound B-3 measured as in Example 1 was 3.6 eV.

### Example 6

### Synthesis of Example Compound B-7

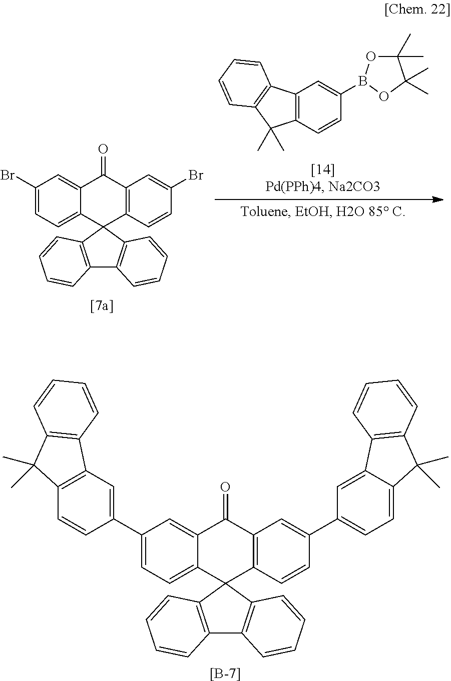

The following reagents and solvents were placed in a 200 mL round-bottomed flask.
[7a]: 1 g (2 mmol)
[14] (fluorenylboronic acid): 1.3 g (4 mmol)
Pd(PPh)4 ((tetrakis(triphenylphosphine)palladium(0)): 0.23 g (0.2 mmol)
Toluene: 50 mL
Ethanol: 20 mL
30 wt % Aqueous sodium carbonate solution: 30 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water, ethanol, and acetone to obtain a crude product. The crude product was dissolved in toluene under heating, subjected to hot filtration, and recrystallized twice with a toluene solvent. The obtained crystals were vacuum dried at 100° C. and purified by sublimation at 10⁻⁴ Pa and 340° C. As a result, 0.62 g (yield: 43%) of high-purity Example Compound B-7 was obtained.

### [MALDI-TOF-MS]

Observed value: m/z=728.7
Calculated value: 728.3
The T₁ energy of Example Compound B-7 measured as in Example 1 was 470 nm on a wavelength basis.

The energy gap of Example Compound B-7 measured as in Example 1 was 3.5 eV.

### Example 7

### Synthesis of Example Compound B-9

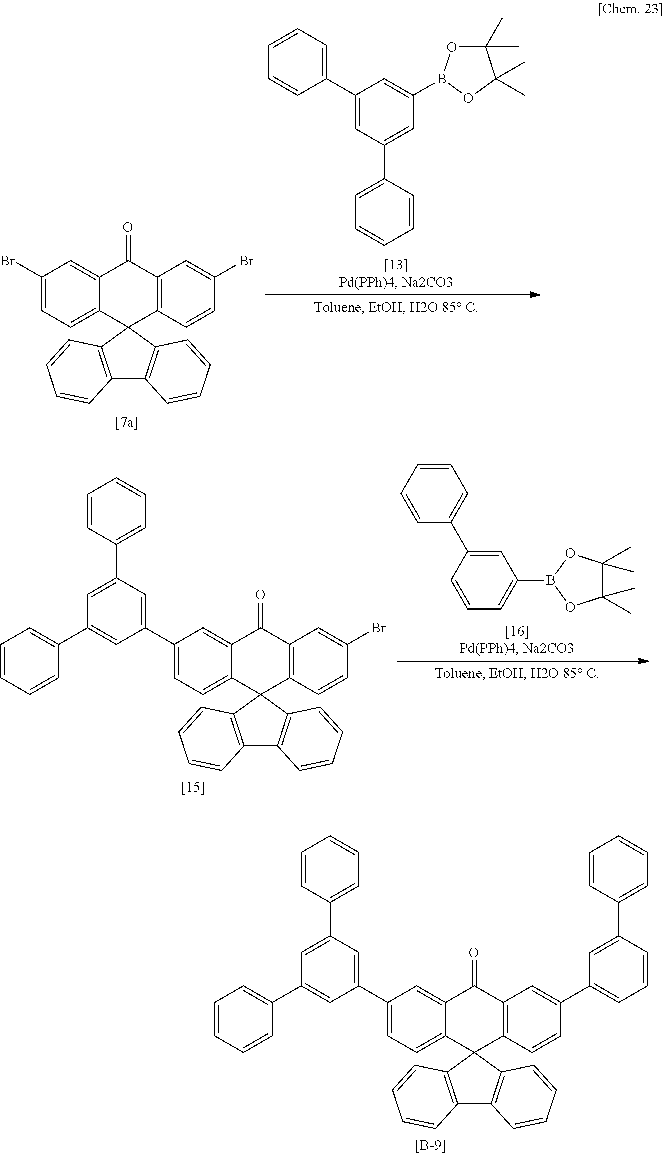

Example Compound B-9, i.e., an asymmetric compound, was synthesized as follows through two reaction stages.

### First Stage

The following reagents and solvents were placed in a 500 mL round-bottomed flask.
[7a]: 5 g (10 mmol)
[13] (terphenylboronic acid): 3.5 g (10 mmol)
Pd(PPh)4 ((tetrakis(triphenylphosphine)palladium(0)): 0.57 g (0.5 mmol)
Toluene: 150 mL
Ethanol: 40 mL
30 wt % Aqueous sodium carbonate solution: 60 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water and ethanol to obtain a crude product. The crude product was purified by column chromatography (filler: silica gel, developing solvent: heptane/ethyl acetate=5/1), dissolved in toluene under heating, subjected to hot filtration, and recrystallized twice with a toluene solvent. The obtained crystals were vacuum dried at 100° C. As a result, 2.9 g (yield: 45%) of an intermediate [15] was obtained. The intermediate [15] was used as a raw material for the reaction of the second stage.

### Second Stage

The following reagents and solvents were placed in a 200 mL round-bottomed flask.
Intermediate [15]: 2 g (3 mmol)
[16] (biphenylboronic acid): 0.86 g (3 mmol)
Pd(PPh)4 (tetrakis(triphenylphosphine)palladium(0)): 0.35 g (0.3 mmol)
Toluene: 80 mL
Ethanol: 20 mL
30 wt % Aqueous sodium carbonate solution: 30 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water, ethanol, and acetone to obtain a crude product. The crude product was dissolved in toluene under heating, subjected to hot filtration, and recrystallized twice with a toluene solvent. The obtained crystals were vacuum dried at 100° C. and purified by sublimation at 10⁻⁴ Pa and 330° C. As a result, 1.1 g (yield: 50%) of high-purity Example Compound B-9 was obtained.

### [MALDI-TOF-MS]

Observed value: m/z=724.9
Calculated value: 724.3
The T₁ energy of Example Compound B-9 measured as in Example 1 was 460 nm on a wavelength basis.

The energy gap of Example Compound B-9 determined as in Example 1 was 3.6 eV.

### Example 8

### Synthesis of Example Compound C-3

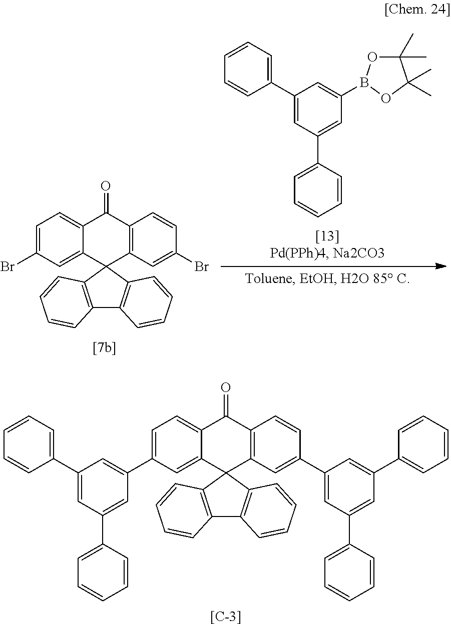

The following reagents and solvents were placed in a 200 mL round-bottomed flask.
[7b]: 1 g (2 mmol)
[13] (terphenylboronic acid): 1.4 g (4 mmol)
Pd(PPh)4(tetrakis(triphenylphosphine)palladium(0)): 0.23 g (0.2 mmol)
Toluene: 50 mL
Ethanol: 20 mL
30 wt % Aqueous sodium carbonate solution: 30 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water, ethanol, and acetone to obtain a crude product. The crude product was dissolved in chlorobenzene under heating, subjected to hot filtration, and recrystallized twice with a chlorobenzene solvent. The obtained crystals were vacuum dried at 100° C. and purified by sublimation at 10⁻⁴ Pa and 325° C. As a result, 0.51 g (yield: 32%) of high-purity Example Compound C-3 was obtained.

### [MALDI-TOF-MS]

Observed value: m/z=800.9
Calculated value: 800.3
The T₁ energy of Example Compound C-3 measured as in Example 1 was 472 nm on a wavelength basis.

The energy gap of Example Compound C-3 measured as in Example 1 was 3.2 eV.

### Example 9

### Synthesis of Example Compound C-9

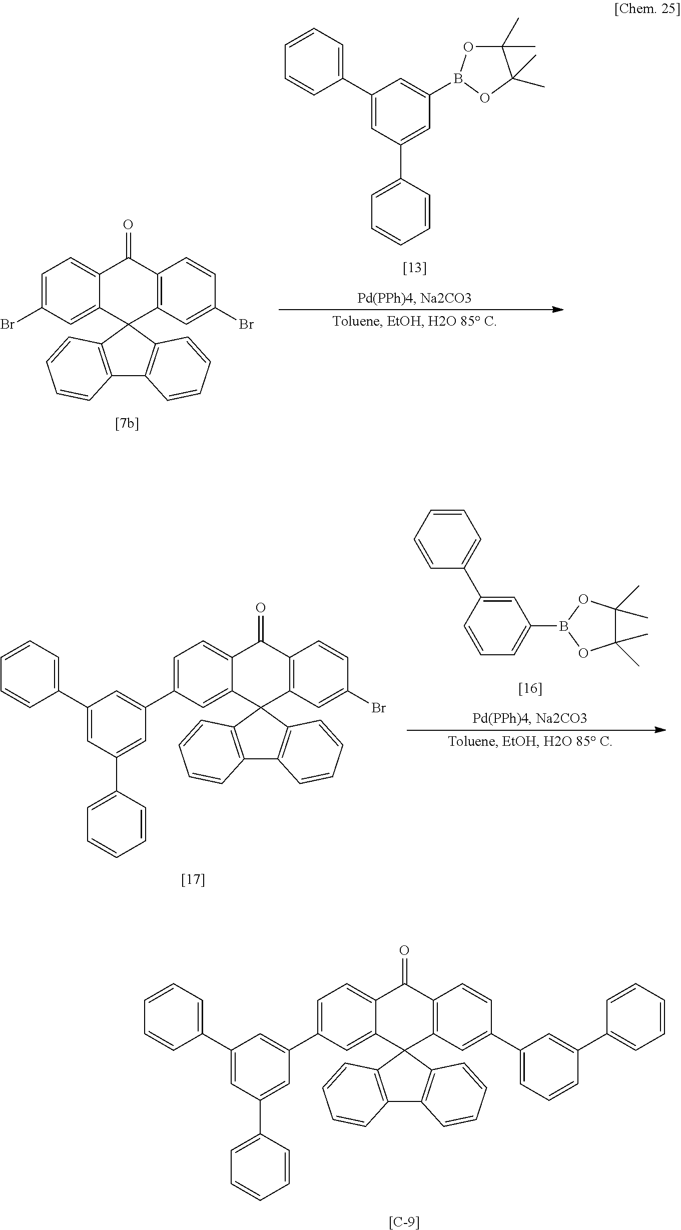

Example compound C-9, i.e., an asymmetric compound, was synthesized as follows through two reaction stages.

### First Stage

The following reagents and solvents were placed in a 500 mL round-bottomed flask.
[7b]: 5 g (10 mmol)
[13] (terphenylboronic acid): 3.5 g (10 mmol)
Pd(PPh)4(tetrakis(triphenylphosphine)palladium(0)): 0.57 g (0.5 mmol)
Toluene: 150 mL
Ethanol: 40 mL
30 wt % Aqueous sodium carbonate solution: 60 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water and ethanol to obtain a crude product. The crude product was purified by column chromatography (filler: silica gel, developing solvent: heptane/ethyl acetate=5/1), dissolved in toluene under heating, subjected to hot filtration, and recrystallized twice with a toluene solvent. The obtained crystals were vacuum dried at 100° C. As a result, 2.1 g (yield: 32%) of an intermediate [17] was obtained. The intermediate [17] was used as a raw material for the reaction of the second stage.

### Second Stage

The following reagents and solvents were placed in a 200 mL round-bottomed flask.
Intermediate [17]: 2 g (3 mmol)
[16] (biphenylboronic acid): 0.86 g (3 mmol)
Pd(PPh)4 (tetrakis(triphenylphosphine)palladium(0)): 0.35 g (0.3 mmol)
Toluene: 80 mL
Ethanol: 20 mL
30 wt % Aqueous sodium carbonate solution: 30 mL

The reaction solution was refluxed for 3 hours under heating and stirring in a nitrogen atmosphere. Upon completion of the reaction, water was added to the reaction solution, followed by stirring. Precipitated crystals were separated by filtration and washed with water, ethanol, and acetone to obtain a crude product. The crude product was dissolved in toluene under heating, subjected to hot filtration, and recrystallized twice with a toluene solvent. The obtained crystals were vacuum dried at 100° C. and purified by sublimation at 10⁻⁴ Pa and 340° C. As a result, 0.84 g (yield: 38%) of high-purity Example Compound C-9 was obtained.

### [MALDI-TOF-MS]

Observed value: m/z=724.9
Calculated value: 724.3
The T₁ energy of Example Compound C-9 measured as in Example 1 was 472 nm on a wavelength basis.

The energy gap of Example Compound C-9 determined as in Example 1 was 3.2 eV.

### Example 10

The LUMO levels of the compounds obtained in Examples 1 to 9 are presented in Table 2. Table 2 shows that the LUMO levels of all compounds were deeper than 2.7 eV.

TABLE 2

| | HOMO(eV) | LUMO(eV) |
|---|---|---|
| A-1 | 6.39 | 2.99 |
| A-3 | 6.38 | 2.99 |
| A-7 | 6.45 | 3.21 |
| B-1 | 6.47 | 2.98 |
| B-3 | 6.47 | 2.99 |
| B-7 | 6.48 | 3.15 |
| B-9 | 6.47 | 3.00 |
| C-3 | 6.35 | 3.25 |
| C-9 | 6.35 | 3.24 |

### Example 11

In Example 11, an organic light-emitting device having an anode/hole transport layer/emission layer/hole blocking layer/electron transport layer/cathode structure, all the layers being sequentially formed on a substrate, was produced by the process below.

Indium tin oxide (ITO) was sputter-deposited on a glass substrate to form a film 120 nm in thickness functioning as an anode. This substrate was used as a transparent conductive support substrate (ITO substrate). Organic compound layers and electrode layers below were continuously formed on the ITO substrate by vacuum vapor deposition under resistive heating in a 10⁻⁵ Pa vacuum chamber. The process was conducted so that the area of the opposing electrodes was 3 mm².
Hole transport layer (40 nm) HTL-1
Emission layer (30 nm)

Host material 1: EML-1

Host material 2: none

Guest material: Ir-1 (10 wt %)
Hole blocking (HB) layer (10 nm) A-3
Electron transport layer (30 nm) ETL-1
Metal electrode layer 1 (0.5 nm) LiF
Metal electrode layer 2 (100 nm) Al

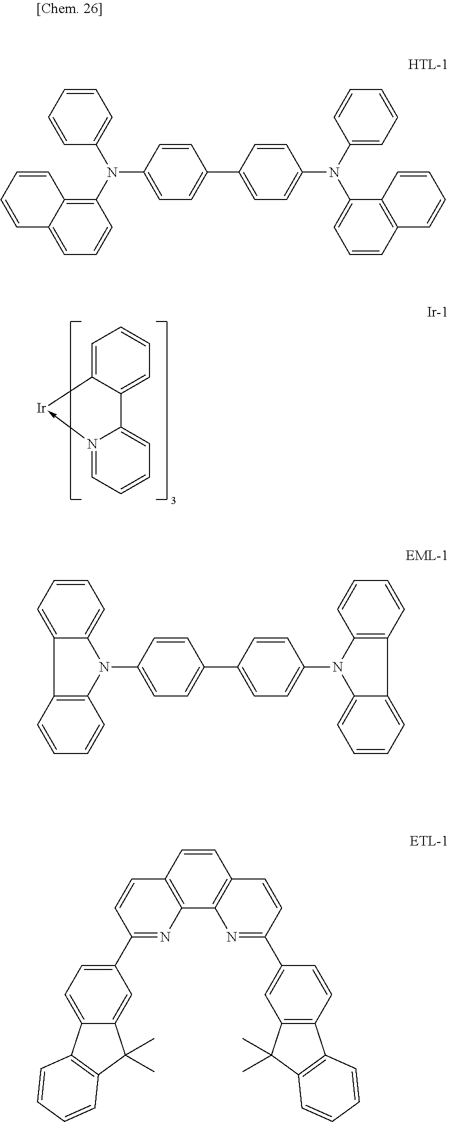

A protective glass plate was placed over the organic light-emitting device in dry air to prevent deterioration caused by adsorption of moisture and sealed with an acrylic resin adhesive. Thus, an organic light-emitting device was produced.

A voltage of 5.5 V was applied to the ITO electrode functioning as a positive electrode and an aluminum electrode functioning as a negative electrode of the resulting organic light-emitting device. The emission efficiency was 55 cd/A and emission of green light with a luminance of 4000 cd/m² was observed. The CIE color coordinate of the device was (x, y)=(0.30, 0.63).

### Examples 12 to 24

In Examples 12 to 24, devices were produced as in Example 11 except that the HB material and the host material 1, the host material 2, and the guest material of the emission layer were changed. Each device was evaluated as in Example 10. The results are shown in Table 3.

TABLE 3

| | HB | Host | Host | Guest | Emission | Voltage | Emission |
|---|---|---|---|---|---|---|---|
| | material | material 1 | material 2 | material | efficiency (cd/A) | (V) | color |
| Example 12 | A-3 | I-3 | None | Ir-1 | 41 | 6.6 | Green |
| Example 13 | A-3 | I-3 | A-3(15%) | Ir-1 | 56 | 5.6 | Green |
| Example 14 | A-7 | I-3 | None | Ir-1 | 40 | 6.4 | Green |
| Example 15 | A-7 | I-3 | A-7(15%) | Ir-1 | 58 | 5.1 | Green |
| Example 16 | B-3 | I-2 | None | Ir-4 | 41 | 6.4 | Green |
| Example 17 | B-3 | I-2 | B-3(15%) | Ir-4 | 55 | 5.5 | Green |
| Example 18 | B7 | I-2 | None | Ir-4 | 40 | 6.6 | Green |
| Example 19 | B-9 | I-2 | None | Ir-4 | 39 | 6.4 | Green |
| Example 20 | C-3 | I-3 | None | Ir-1 | 42 | 6.5 | Green |
| Example 21 | C-3 | I-3 | C-3(15%) | Ir-1 | 58 | 5.4 | Green |
| Example 22 | C-9 | I-3 | None | Ir-1 | 38 | 6.2 | Green |
| Example 23 | A-1 | I-5 | None | Ir-11 | 9 | 6.7 | Blue |
| Example 24 | B-1 | I-5 | None | Ir-13 | 11 | 6.8 | Blue |

The results show that when the spiro(anthracene-9,9′-fluoren)-10-one compound is used as an electron transport material or an emission layer material of a phosphorescent organic light-emitting device, high emission efficiency can be achieved.

### Examples 25 and 26 and Comparative Examples 1, 2, and 3

The structural formulae of the compounds of Examples 25 and 26 and Comparative Examples 1, 2, and 3 are as follows.

[Structural formulae of compounds used in Examples 25 and 26]

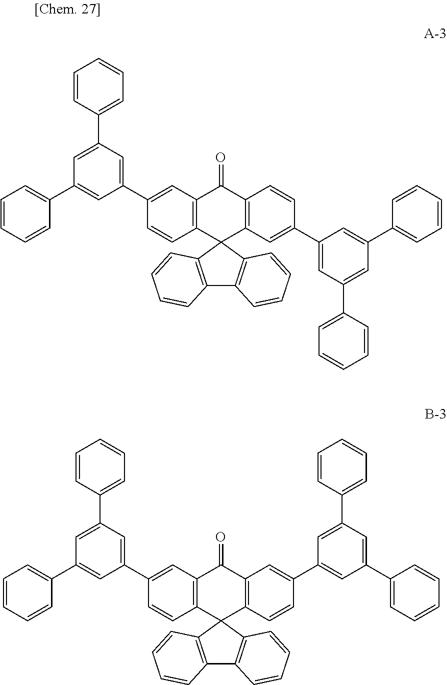 [SMILES: O=C1c2cc(-c3cc(-c4ccccc4)cc(-c4ccccc4)c3)ccc2C2(c3cc(-c4cc(-c5ccccc5)cc(-c5ccccc5)c4)ccc31)c1ccccc1-c1ccccc12 ; O=C1c2cc(-c3cc(-c4ccccc4)cc(-c4ccccc4)c3)ccc2C2(c3ccc(-c4cc(-c5ccccc5)cc(-c5ccccc5)c4)cc31)c1ccccc1-c1ccccc12]

[Structural formulae of compounds used in Comparative Examples 1 to 3]

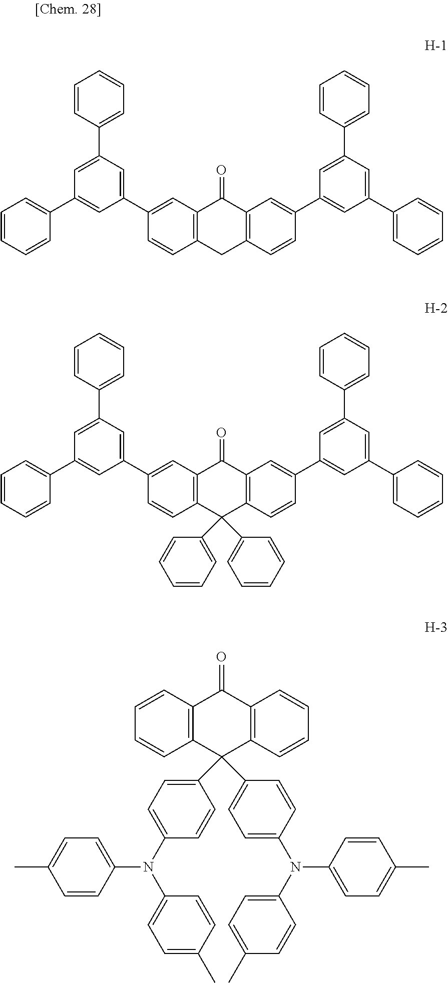

### Structure and Stability

The compound H-1 of Comparative Example 1 is a compound having the 10-position of the anthrone skeleton substituted with hydrogen. As discussed earlier, the stability decreases (the structure turns into anthracene) when the 10-position of the anthrone skeleton is substituted with hydrogen.

The compound H-2 of Comparative Example 2 and the compound H-3 of Comparative Example 3 have the 10-position of the anthrone skeleton substituted with two aryl groups (phenyl groups). Since the two aryl groups can rotate separately, the stability of the basic skeleton is low.

That the difference in the structure of the basic skeleton affects the stability (lifetime) of the organic light-emitting devices was confirmed through the evaluation below.

### Comparison of LUMO Level and Electron Mobility

The electron mobility of the compounds A-3, B-3, and H-3 of Examples 25 and 26 and Comparative Example 3 is presented in Table 4. The electron mobility of A-3 and B-3 is two orders of magnitude higher than the hole mobility thereof. In contrast, H-3 has the 10-position of the anthrone skeleton substituted with an arylamine group having a hole transport property and thus exhibits electron mobility not higher than the hole mobility. Moreover, the electron transport property of H-3 tends to be inhibited (electron mobility tends to be low). The evaluation confirms that the stability (lifetime) of the light-emitting device using H-3 is significantly deteriorated due to this difference.

The mobility was determined by forming a thin film (1 to 3 μm in thickness) of each compound by a sublimation method on an ITO substrate to form an evaluation sample and measuring the mobility of the sample by a time-of-flight technique (analyzer produced by Sumitomo Heavy Industries, Ltd., Mechatronics division).

TABLE 4

| | Electron mobility/hole mobility |
|---|---|
| A-3 | 10-3 (cm2/Vsec)/10-5 (cm2/Vsec) |
| B-3 | 10-3 (cm2/Vsec)/10-5 (cm2/Vsec) |
| H-3 | 10-4 (cm2/Vsec)/10-4 (cm2/Vsec) |

### Comparison of Luminance Half Life of Organic Light-emitting Device

In Examples 25 and 26 and Comparative Examples 1 to 3, devices were produced as in Example 11 except that the hole blocking material and the host material 1, the host material 2, and the guest material of the emission layer were changed. The luminance half life of each organic light-emitting device at a current value of 40 mA/cm² was measured to evaluate the stability of the device. The results are presented in Table 5. In the table, the hole blocking material is denoted as “HB material”.

TABLE 5

| | HB | Host | Host | Guest | Luminance |
|---|---|---|---|---|---|
| | material | material 1 | material 2 | material | half life (h) |
| Example 25 | A-3 | I-3 | None | Ir-1 | 305 |
| Example 26 | B-3 | I-3 | None | Ir-1 | 325 |
| Comparative | H-1 | I-3 | None | Ir-1 | 65 |
| Example 1 | | | | | |
| Comparative | H-2 | I-3 | None | Ir-1 | 95 |
| Example 2 | | | | | |
| Comparative | H-3 | I-3 | None | Ir-1 | 30 |
| Example 3 | | | | | |

The spiro(anthracene-9,9′-fluoren)-10-one compounds of the embodiments extended the luminance half life of a phosphorescent organic light-emitting device compared to the compounds of Comparative Examples. This is because the spiro(anthracene-9,9′-fluoren)-10-one compound having a spiro structure performed more stably in an excited state.

The spiro(anthracene-9,9′-fluoren)-10-one compound according to embodiments of the present invention has high T₁ energy, a deep LUMO level, and high electron mobility. When the spiro(anthracene-9,9′-fluoren)-10-one compound is used in an organic light-emitting device, high emission efficiency and stability resistant to deterioration can be achieved.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-158569, filed Jul. 13, 2010, which is hereby incorporated by reference herein in its entirety.

1. A spiro(anthracene-9,9′-fluoren)-10-one compound represented by general formula [1]: 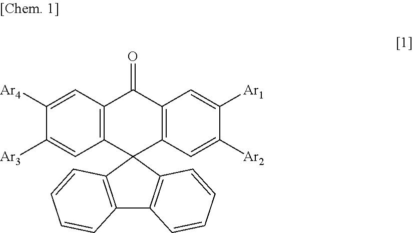
where Ar₁ and Ar₂ each independently denote a hydrogen atom, a phenyl group, a biphenyl group, a terphenyl group, a dimethylfluorenyl group, a triphenylene group, a dibenzofuran group, or a dibenzothiophene group, one of Ar₁ and Ar₂ denoting a hydrogen atom, and Ar₃ and Ar₄ each independently denote a hydrogen atom, a phenyl group, a biphenyl group, a terphenyl group, a dimethylfluorenyl group, a triphenylene group, a dibenzofuran group, or a dibenzothiophene group, one of Ar₃ and Ar₄ denoting a hydrogen atom.

2. An organic light-emitting device comprising:
an anode;
a cathode; and
a first organic compound layer disposed between the anode and the cathode, the organic compound layer containing the spiro(anthracene-9,9′-fluoren)-10-one compound according to claim 1.

3. The organic light-emitting device according to claim 2, further comprising:
a second organic compound layer that serves as an emission layer, wherein the first organic compound layer is in contact with a cathode-side of the second organic compound layer that serves as the emission layer.

4. The organic light-emitting device according to claim 3, wherein the emission layer contains a host material and a guest material, the host material including a first host material and a second host material, and the second host material is the spiro(anthracene-9,9′-fluoren)-10-one compound.

5. The organic light-emitting device according to claim 4, wherein the guest material is a phosphorescent material.

6. The organic light-emitting device according to claim 5, wherein the phosphorescent material is an iridium complex.

7. The organic light-emitting device according to claim 3, wherein the organic light-emitting device emits green light.

8. An image display apparatus comprising:
the organic light-emitting device according to claim 2; and
a switching device connected to the organic light-emitting device.
